(19) United States
(12) Patent Application Publication
(10) Pub. No.: US 2010/0196880 A1
(43) Pub. Date: Aug. 5, 2010

(54) COMPOSITIONS AND MULTIPLEX ASSAYS FOR MEASURING BIOLOGICAL MEDIATORS OF PHYSIOLOGICAL HEALTH

(76) Inventors: Ebenezer Satyaraj, Wildwood, MO (US); Steven S. Hannah, Chesterfield, MO (US)

Correspondence Address: WENDELL RAY GUFFEY;NESTLE PURINA PETCARE GLOBAL RESOURCES, INC., 1 CHECKERBOARD SQUARE, 11-T, ST. LOUIS, MO, 63164, US

(21) Appl. No.: 12/311,542

(22) PCT Filed: Oct. 4, 2007
(86) PCT No.: PCT/US2007/021451

§ 371 Date: Apr. 2, 2009

Related U.S. Application Data
(60) Provisional application No. 60/849,928, filed on Oct. 6, 2006.

(51) Int. Cl. C12Q 1/68; C07H 21/04

(52) U.S. Cl. 435/6; 536/24.31

(57) ABSTRACT

Multiplex assays are provided including panels of probes for development of multiplex assays capable of simultaneously measuring multiple biologically-relevant proteins using very small quantities of biological samples to rapidly assess the health status of animals, especially companion animals, as well as to formulate nutritional regimens for improving the health status of animals. The probes are provided as are methods for using them to assess the health status of animals, as well as their responses to therapeutic or nutritional interventions therein.

### CROSS REFERENCE TO RELATED APPLICATIONS

This application claims priority to U.S. Provisional Application Ser. No. 60/849,928 filed Oct. 6, 2006, and to PCT Application No. PCT/US2007/021451, the disclosure of which is incorporated herein by this reference.

### BACKGROUND OF THE INVENTION

1. Field of the Invention

This invention relates generally to facile assays for determining the health status of animals and particularly to multiplex assays involving the measurement of cytokines, hormones, and adipokines to access animal health status and the effect of nutrition on health status.

2. Description of Related Art

Cytokines, adipokines, and hormones are among the principal biological mediators orchestrating physiological response to stimuli and stress and are therefore useful as ‘signatures’ of health status and/or indicators of disease. Evaluating changes, both static and temporal, in these mediators provides some understanding of the response of a biological system or organism to stressors.

Multiplex analysis is convenient and allows the study of multiple analytes using instruments such as the Luminex xMAP platform. Such multiplex analysis enables simultaneous quantitative measurement of up to 100 analytes in a single assay. Such assays are thus suitable for the accumulation of the data required for, and the nature of the samples involved in, the study of biological mediators such as those described herein.

Although the techniques for measuring many biological mediators are known, there remains a need for panels of probes for development of multiplex assays capable of simultaneously measuring multiple biologically-relevant proteins using very small quantities of biological samples to rapidly assess the health status of animals, especially companion animals, as well as to formulate nutritional regimens for improving the health status of animals. In particular, there is a need for the probes and methods to assess the health status of animals and to therapeutic or nutritional interventions useful to affect the improve animal health.

### SUMMARY OF THE INVENTION

It is, therefore, an object of the present invention to provide panels of probes for development of multiplex assays capable of simultaneously measuring multiple biologically-relevant proteins using very small quantities of biological samples.

It is another object of the present invention to provide methods for rapidly assess the health status of animals.

It is another object of the present invention to provide nutritional regimens for improving the health status of animals.

It is a further object of the invention to provide articles of manufacture in the form of kits that contain combinations of the multiplex assays of the present invention and instruction for how to use the assays for various purposes.

One or more of these and other objects are achieved using novel collections of detectable molecular probes for determining the activity, presence, or expression of each of a predetermined set of analytes in a single sample. The set of analytes comprise at least one cytokine or gene therefor, one chemokine or gene therefor, one hormone or gene therefor, and one adipokine or gene therefor and, for each analyte in the set, the collection of molecular probes comprises at least one probe suitable for detecting the activity, presence, or expression of that analyte. In some embodiments, the set of analytes further comprises one or more neuronal growth factors or genes therefor, growth factors other than neuronal growth factors or genes therefor, soluble receptors or genes therefor, or combinations thereof.

Other and further objects, features, and advantages of the present invention will be readily apparent to those skilled in the art.

### BRIEF DESCRIPTION OF THE DRAWINGS

FIG. 1 Diagrammatic presentation of Metabolic Syndrome showing the central role that insulin resistance holds and ultimate risk of heart disease, stroke, and/or inflammation.

FIG. 2 Cytokines response to LPS stimulation: PBMCs were cultured with different conc. of LPS for 3, 6, or 18 hrs. C/S were assayed for cytokines. Data for IL-6 [A], TNFα [B], IL-18 [C] and IL-8 [D] are shown. X-axis LPS dose ng/ml, Y-axis is Mean Fluorescence Intensity [MFI] or [MF] and Z-axis represents time-point.

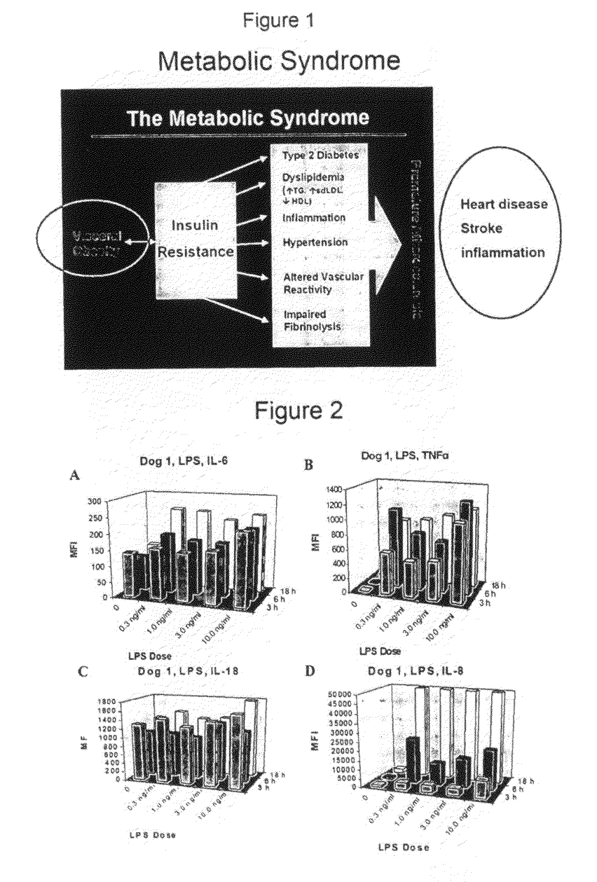

### DETAILED DESCRIPTION OF THE INVENTION

### Definitions

The term “activity” of a gene encompasses any measure that it is related to the central biological roles played by a gene For example, measurement of the transcription of a gene, or measure either at static time points or in real time of the abundance of an RNA species transcribed therefrom. The skilled artisan will appreciate that a measure of a gene's “activity” as used herein can also include measurements in specific tissues, cell-types, or organs, of the amount of mRNA produced from a particular gene, whether in real time or not. Similarly, the skilled artisan will also appreciate that protein analytes can be measured in a variety of useful ways. Measurement of protein and other analytes can include the presence or absence of the analyte, activity (e.g. enzyme activity or other biological activity), binding properties, half-life, turn-over, or other measurable attributes of the analyte.

The term “analyte” includes proteins “expressed from a gene” in the form of native proteins as they are translated in the cell and proteins having post-translational translocation, processing, modifications, and the like. Thus, in some cases protein analytes may be truncated after translation, or for example may be phosphorylated, or have other modifications such as to the backbone or to the side chain of any amino acid residue. The term analyte also includes metabolic derivatives of such proteins, and complexes, whether active or not, of one or more proteins with one or more other substituents found in a cell. In preferred embodiments, the analytes are protein or peptides and the probes are antibodies. Each antibody in the collection of probes specifically recognizes only one protein in the set, and there is at least one such antibody in the collection for each protein in the selected set. The skilled artisan will appreciate that once a set of such proteins has been selected, if the amino acid sequences of those proteins are known, it is often within the skill of the ordinary practitioner to design a set of nucleic acid or other probes that correspond to the genes or mRNAs from which those proteins are expressed. Accordingly, such applications are also useful herein.

The term “animal” means any animal having cytokines, hormones, adipokines, neuronal growth factors, growth factors other than a neuronal growth factors, or soluble receptors useful in the present invention, including, but not limited to, human, avian, bovine, canine, equine, feline, hicrine, murine, ovine, and porcine animals, preferably humans, murines simians, canines, and felines.

The term “collection” in referring to a group of molecular probes means a plurality. Typically the plurality has no limit, although collections of 100 or fewer probes are preferred.

The term “molecular probe(s)” means any molecule that can be used to detect the presence or activity of a gene, its corresponding RNA, or its protein expression product.

The term “panel” is synonymous with the term collection. The term panel is sometimes more descriptive of the use of the collection in screening samples. A collection as used herein is preferably, but not necessarily, used for detecting a corresponding set of analytes in multiplex fashion, i.e., all of the results for each of the plurality of probes are obtained from a single reaction or assay vessel. A collection of probes typically corresponds to a set of analytes, wherein each of the plurality of probes corresponds to a particular analyte in the set. In certain embodiments, the analytes are genes or gene products (proteins) and the probes allow the measurement of the activity or expression of each of the genes (or their expression products) in the group (or set). More preferably, the analytes are proteins expressed from the gene.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual assay components physically associated such that they are considered a unit for manufacture, distribution, sale, or use.

The term “virtual package” means that the components of a kit are associated by directions on one or more physical or virtual kit components instructing the user how to obtain the other components, e.g., in a package containing one component and directions instructing the user to go to a website, contact a recorded message, view a visual message, or contact a caregiver or instructor to obtain instructions on how to use the kit.

### THE INVENTION

In one aspect, the present invention provides a collection of detectable molecular probes for determining in a single sample, the activity, presence, or expression of each of a predetermined set of analytes. The set of analytes comprises at least one cytokine or gene therefor, one chemokine or gene therefor, one hormone or gene therefor, and one adipokine or gene therefor. For each analyte in the set, the collection of molecular probes comprises at least one probe suitable for detecting the activity, presence, or expression of that analyte. These collections of probes are intended for use in a detection method that is preferably conducted in a convenient assay format in a single reaction vessel. In conjunction with appropriate pattern recognition and pathway analysis techniques these panels help predict or evaluate functional outcomes of physiological stressors and interventions.

Preferably, the molecular probes comprise protein, nucleic acid, or combinations thereof but may comprise small molecules or other compounds and structures or combinations thereof. Examples of probes comprising protein include antibodies, antibody fragments, receptors, binding proteins, enzymes, and the like. They may be used to probe for not only protein analytes, but a variety of other analytes. Nucleic acid probes include those whose specificity arises through complementary Watson-Crick-type base-pairing, as well as those whose specificity arises from or includes other interactions. For example, aptamers, nucleic acids that can be designed to specifically recognize certain analytes, such as proteins or other molecules, are useful herein. Nucleic acid enzymes such as DNAzymes and ribozymes are known in the art, have known specificities, and are useful as probes herein. Probes may also comprise ligands for binding molecules and receptors. The use of all such molecules as probes for analytes, such as the expression products of genes, or even the genes themselves, is known in the art, and thus the skilled artisan will appreciate how to select and adapt such molecules for use herein.

Probes can be man-made or isolated from nature. In preferred embodiments, the molecular probes are antibodies. In one embodiment, each antibody in a collection can specifically recognize and thus serve to identify, one protein analyte in a corresponding set of proteins. Probes may be used in any convenient format, such as in solution or suspension. In certain embodiments, they may bound to a substrate, such as a carrier or a bead, or placed in an array, microarray, or the like so as to create a useful, convenient, and/or informative assay system.

The predetermined set of analytes in certain embodiments is a set of genes, or a set or proteins. In certain embodiments a set of protein analytes is selected and assays based on a corresponding set of genes or mRNAs are developed. The set of analytes is selected on a rational basis, based on its relationship to the information to be obtained from the panel. In a preferred embodiment, the set of analytes is selected based on the relationship of each analyte to the health status of an individual.

In one embodiment, the set of analytes is a set of proteins comprising at least one cytokine, one chemokine, one hormone, and one adipokine. For each analyte in the set, the collection of molecular probes comprises at least one probe suitable for detecting the presence, activity or expression of that analyte—i.e. there is a corresponding probe for each analyte in the set whose presence, activity, or expression is to be determined. In a further embodiment, wherein the collection of molecular probes is the foregoing set of protein analytes, the set of analytes further comprises one or more of at least one other type of probiotic organism

In another embodiment, the set of analytes comprises a set of genes with at least one gene encoding a cytokine, one gene encoding a chemokine, one encoding a hormone, and one, an adipokine. In one embodiment, the set of gene analytes further comprises at least one gene encoding a neuronal growth factor, at least one gene encoding another growth factor (i.e. growth factors other than neuronal growth factors), or at least one gene encoding soluble receptors, or any combination thereof.

While there is no actual limit to the number of probes that can be present in a single collection in accordance herewith, it is preferred that an upper limit is 100 probes per collection in certain embodiments. Smaller collections of probes are also suitable. For example panels of about 90-100, 80-90, 70-80 or 60-70 probes are all suitable for use herein. Similarly, collections of about 10-20, 20-30, 30-40, 40-50 or 50-60 are also suitable for use. Other specific numbers of probes from 4 to 100 are also included herein, although not specifically enumerated, as are all possible ranges of from 4-100 probes included herein, though not specifically enumerated. Ranges of probes are particularly useful where some redundancy may be initially desirable, and later found to be unnecessary, or alternatively, where an additional probe may be determined to be useful to include with a particular collection as more about its role in vivo is discovered or appreciated. In other embodiments, for example where the probes may be bound to an array or microarray, it may be useful, and thus preferable, to exceed 100 probes per collection.

In one embodiment, the collection of molecular probes comprises detectable probes for detecting a protein (i.e., an encoded gene product) of each of a set of genes thereby determining the expression of each gene in the set. In such embodiments, preferably each probe is specific for detecting the encoded protein for one gene in the set. In some embodiments, a degree of cross-reactivity may be experimentally acceptable. This is particularly true where the probes are themselves biological molecules, such as antibodies. The cross-reactivity of certain antibodies is recognized in the art. The skilled artisan will appreciate that cross-reactivity of antibodies to closely-related antigens, such as some proteins can create problems, especially if severe. In one embodiment, the cross-reactivity is minimized or eliminated through the use of monospecific antibodies, such as highly-purified antibodies, or monoclonal antibodies to specific epitopes that are immunologically distinguishable. In another embodiment, the cross-reactivity is distinguishable from the intended activity based on binding properties such as binding constants, or a measure of binding strength, or the like. In another embodiment, the use of proper and careful controls, or other means, such as computer analysis of results allows the correction of experimental data for cross-reactivity of certain types.

While there are many choices for the set of analytes, as discussed herein, a rational approach to the selection of analytes is preferred. The collection of molecular probes will preferably be designed or selected with the goal of the intended use in mind. To that end, the set of analytes is predetermined via a rational approach which rests on the known, predicted, or herein discovered relationships between the presence or activity of certain analytes to various aspects of health of an animal. Factors such as the state of inflammation in an animal and the relative presence of certain hormones and other biochemical signals or signal conductors can help to provide detailed information linked to the health status of an animal.

Thus, in one embodiment, the set of analytes is a set of proteins that comprises one or more cytokines. Preferably, the cytokine includes one or more of interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III.

In various embodiments, the set of proteins comprises one or more chemokines, for example, B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1. Of course, the set may comprise the foregoing chemokines in addition to the cytokines exemplified herein.

In various embodiments, the set of proteins comprises one or more hormones. Preferred are the hormones prolactin, insulin-like growth factor binding protein 2, leptin, insulin, resistin, adiponectin, glucagon, glucagon-related peptide 1, or PYY. The skilled artisan will appreciate that other hormones may be selected. Preferably, the activity or the presence of the hormones is affected by a selected nutritional regimen as discussed below, or there is a relationship between the activity or presence of the hormone to the health status of an animal. As herein, and for each of the categories exemplified herein as within the rational predetermination of the set of proteins, the inclusion of certain hormones does not preclude and may be in addition to the inclusion of other molecules for example cytokines, chemokines, adipokines, and others described herein.

In another embodiment, the set of proteins comprises one or more adipokines, including but not limited to monocyte chemotactic protein 1, leptin, resistin, adiponectin, IL-6, TNF-alpha, or thrombin-activatable fibrinolysis inhibitor.

In certain embodiments, the predetermined set of protein analytes further comprises one or of a neuronal growth factor, a growth factor other than a neuronal growth factor, or a soluble receptor, or combinations thereof.

Neuronal growth factors for use herein include but not limited to ciliary neurotrophic factor, glial cell line derived neurotrophic factor, brain-derived neurotrophic factor, neurotrophin 3, neurotrophin 4, or beta-nerve growth factor.

Growth factors are preferably selected from angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2.

The soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cytotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4, and others, are all useful herein.

In another preferred embodiment, the set of analytes is a set of genes that comprises one or more genes encoding one or more cytokines. Preferably, the encoded cytokines include one or more of the cytokines enumerated herein: interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III.

The set of genes comprises, in certain embodiments, one or more genes encoding one or more chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1. Of course, the set may comprise one or more genes for the foregoing chemokines in addition to those genes for the cytokines exemplified herein.

In various embodiments, the set of genes comprises one or more genes encoding various hormones. Preferred are genes encoding the hormones prolactin, insulin-like growth factor binding protein 2, leptin, insulin, resistin, adiponectin, glucagon, glucagon-related peptide 1, or PYY. Preferably, there is a relationship between the activity or expression of the encoded hormone to the health status of an animal. The inclusion in the set of analytes of one or more genes encoding hormones does not preclude the inclusion of one or more genes encoding other molecules, for example, cytokines, chemokines, adipokines, and others described herein.

In another embodiment, the set of genes comprises one or more genes encoding one or more of the adipokines monocyte chemotactic protein 1, leptin, resistin, adiponectin, IL-6, TNF-alpha, or thrombin-activatable fibrinolysis inhibitor. Genes for other adipokines are also useful herein.

In certain embodiments, the predetermined set of genes further comprises one or more genes encoding a neuronal growth factor, a growth factor other than a neuronal growth factor, or a soluble receptor. Preferred neuronal growth factors, growth factors other than neuronal growth factors, and soluble receptors include but are not limited to those enumerated herein for the set of proteins. Other such molecules are also contemplated for use herein.

In various embodiments, the set of analytes comprises one or more primary or secondary metabolic products. Thus, in one embodiment, the set of analytes includes one or more eicosanoids, a class of oxygenated hydrophobic molecules that largely function as autocrine and paracrine mediators of biological functions. For example, leukotrienes are known to serve as agents in the inflammatory response. Some have a chemotactic effect on migrating neutrophils, and as such help to bring necessary cells to the involved tissue. Leukotrienes also are powerful vasoconstrictors, particularly of venules. They function in bronchoconstriction, and can also increase vascular permeability. Leukotrienes suitable for use herein include but are not limited to LTA4, LTB4, LTC4, LTD4, LTE4, and LTF4. Other eiscosanoids suitable as analytes for use herein include thromboxanes and the prostaglandin H derivatives, prostanoids. Still other eiscosanoid compounds such as the resolvins, isofurans, isoprostanes, lipoxins, epoxyeicosatrienoic acids (EETs), neuroprotectin D and 20-carbon endocannabinoids may be suitable for use herein as analytes. In addition, eicosanoid receptors, such as the leukotriene receptors CysLT1 (Cysteinyl leukotriene receptor type 1), CysLT2 (Cysteinyl leukotriene receptor type 2), and BLT1 (Leukotriene B4 receptor); the prostanoids receptors PGD2: DP-(PGD2), PGE2: EP1-(PGE2), EP2-(PGE2), EP3-(PGE2), EP4-(PGE2), PGF2α: FP-(PGF2α), PGI2 (prostacyclin): IP-(PGI2), and TXA2 (thromboxane): TP-(TXA2) are also useful as analytes in any of the aspects or embodiments herein.

In a preferred embodiment, the collection of molecular probes comprises a specific probe for each of IL-2, IL-4, IL-6, IL-7, IL-8, IL-10, IL-18, IFN γ, IP-10, TNF-α, MCP-1, GLP-1, glucagon, insulin, adiponectin, and resistin. The collection of probes further comprises specific probes for one or more of IL-15, KC, and leptin in certain embodiments.

In another preferred embodiment, the collection of molecular probes comprise probes using canine specific molecules suitable for producing for a canine assay: Panel; Cytokine/chemokine; Analyte: GMCSF, IL-2, IL-4, IL-6, IL-7, IL-8, IL-10, IL-18, IFNγ, IP-10, TNF-α, MCP-1, IL-15, KC. Panel; Endocrine; Analyte: GLP-1, Glucagon, Insulin, Leptin. Panel; Adipokine; Analyte: Adiponectin, Resistin. In another, the collection of molecular probes comprise probes using feline molecules suitable for producing a feline assay: Panel; Cytokine/chemokine; Analyte: GMCSF, IL-2, IL-4, IL-6, IL-1β, IL-8, IL-10, IL-18, IFNγ, Fas, TNF-α, MCP-1, Flt-3 ligand. Panel; Endocrine; Analyte: GLP-1, Glucagon, Insulin, Leptin. Panel; Adipokine; Analyte: Adiponectin, Resistin.

In one embodiment, the molecular probes are capable of detecting the presence, activity, expression, or the like of each of the analytes in the predetermined set of analytes. Preferably the set of analytes are the sets of proteins and/or genes set forth herein. More preferably the genes are from a human, a simian, a canine, or a feline. In preferred embodiments, the collection of probes is specific for detecting the presence or activity of a set of canine or feline proteins.

In one embodiment, the collection of molecular probes is a plurality of antibodies specific for measuring or detecting protein analytes (i.e. the expression products from a set of genes) from a canine. Preferably, the probes provide a quantitative determination of the amount of expression of each protein, such as the amount of each specific protein present in the sample. In certain embodiments, the results are more qualitative providing information as to relative amounts, for example indicating differences or changes from sample to sample, or changes over a time course in samples from an individual. In other embodiments, analogous measurements are obtain for the set of genes corresponding to the selected proteins.

In various embodiments, for the collection of molecular probes described, each probe is attached to a matrix, or support, wherein each such attached probe remains capable of providing a quantitative determination of the amount of activity or presence of each of a set of analytes in a sample brought into contact with the matrix. The skilled artisan will appreciate that probes of the type described herein can be attached, immobilized, or supported in a variety of ways to allow for more facile assays to be developed. In such embodiments an entire collection of probes could attached to a single matrix and distinctions can be made for example through spatial separation of a set of proteins (or expressed gene products), for example, electrophoretically, or though the detection of discrete or individually detectable signals corresponding to each of the probes. In some embodiments, the probes are spatially arranged as a array or microarray. The use of arrays such as ‘chips’ and the like is also useful herein. Alternatively, each probe may be attached to a separate matrix, use of such probes is also known in the art. Examples of matrices to which probes can be attached include various membranes, polymers. supports, beads, chips, arrays, assay wells and the like. Assays methods such as ELISA and FACS can be used to detect such probes that are attached to a matrix. Ideally, such attachment can aid in the design of multiplex assays wherein the plurality of genes in the set of predetermined genes can be assayed in a single reaction vessel. Attachment of the molecular probes to various types of polymeric or glass beads can provide a simple format for developing a multiplex assay using, for example differential labeling and FACS as a method for distinguishing one probe from another. Thus, in one embodiment, the collection of molecular probes is provided wherein each probe is attached to a separate matrix, and each probe is independently detectable from each other probe in the collection. The probes can also be attached either covalently, or through other means, to various molecular components such as signals and other molecules that can, for example, either amplify or quench signals, or facilitate assay development, as is useful. Alternatively, such signal enhancers or quenchers, and other molecules may be used in conjunction with the probes while not physically nor chemically attached thereto.

The various cytokines, chemokines, hormones, and adipokines, as well neuronal growth factors, growth factors, and soluble receptors are sometimes referred to herein by their abbreviations or other shorthand nomenclature reference. Shown below is a list of such abbreviations and other reference names shown as “Reference; Systematic (in parenthesis when available); and Name:”.

Cytokines. IFN-α; Interferon alpha: IFN-γ; Interferon gamma: IL-12 (p40); Interleukin 12 p40: IL-18; Interleukin 18: IFN-β; Interferon beta: IFN-ω; Interferon omega: Lymphotoxin βR; (TNFRSF3); Lymphotoxin beta R: Lymphotactin (Lptn); (XCL1); Lymphotoxin: IL-6; Interleukin 6: IL-8; (CXCL8); Interleukin 8: TNF-α; Tumor necrosis factor alpha: IL-4; Interleukin 4: IL-10; Interleukin 10: TGF-β1; Transforming growth factor beta-1: TNF-β; Tumor necrosis factor beta: IL-3; Interleukin 3: IL-5; Interleukin 5: IL-7; Interleukin 7: IL-13; Interleukin 13: IL-15; Interleukin 15: IL-1α; Interleukin 1 alpha: IL-1β; Interleukin 1 beta: IL-2; Interleukin 2: IL-11; Interleukin 11: IL-12 (p70); Interleukin 12 p70: IL-16; Interleukin 16: IL-17; Interleukin 17: RANTES; (CCL5); Regulated upon Activation: Normal T Expressed and presumably Secreted: IL-21; Interleukin 21: IL-9; Interleukin 9: and TGF-β RIII; Transforming growth factor beta receptor III.

Chemokines. BLC (BCA-1); B-lymphocyte chemoattractant: ENA-78; (CXCL5); Epithelial cell-derived neutrophil-activating peptide: Eot; (CCL11); Eotaxin: Eot-2; (CCL24); Eotaxin-2: MCP-2; (CCL8); Monocyte chemotactic protein 2: MCP-3; (CCL7); Monocyte chemotactic protein 3: MIF; Macrophage migration inhibitory factor: MIP-1α; (CCL3); Macrophage inflammatory protein 1 alpha: MPIF; Myeloid progenitor inhibitory factor 1: MSP; Macrophage stimulating protein: GCP-2; (CXCL6); Granulocyte Chemotactic Protein 2: 1-309; (CCL1); None: IP-10; (CXCL10); Interferon gamma inducible protein 10: LIF; Leukemia inhibitory factor: M-CSF; Macrophage colony stimulating factor: MCP-1; (CCL2); Monocyte chemotactic protein 1: MDC; (CCL22); Macrophage derived chemokine: MIG; (CXCL9); None: MIP-1β; (CCL4); Macrophage inflammatory protein 1 beta: MIP-1δ; (CCL15); Macrophage inflammatory protein 1 delta: NAP-2; Neutrophil Activating Peptide 2: PARC; Pulmonary and activation-regulated chemokine: SDF-1α; Stromal cell derived factor alpha: TARC; (CCL17); Thymus and activation regulated chemokine: BTC; Betacellulin: 6Ckine; (CCL21); 6Ckine: FGF acid (FGF-1); Fibroblast growth factor acidic: Fractalkine; (CX3CL1); Fractalkine: HCC-1; (CCL14); Hemofiltrate CC Chemokine 1: MCP-4; (CCL13); Monocyte Chemotactic Protein 4: MIP-3β; (CCL19); Macrophage inflammatory protein 3 beta: PF4; (CXCL4); Platelet factor 4: RANK; (TNFRSF11A); Receptor Activator of NF-kappa-B: CTACK; (CCL27); Cutaneous T-cell Attracting Chemokine Eot-3; (CCL26); Eotaxin-3: FGF-4; Fibroblast growth factor-4: Follistatin; Follistatin: GRO-γ; (CXCL3); Growth Related Oncogene gamma: I-TAC; (CXCL11); Interferon gamma-inducible T Cell alpha Chemoattractant: sLIF-Rα (gp190); Leukemia Inhibitory Factor receptor alpha: Midkine; Midkine: MIP-3α; (CCL20); Macrophage inflammatory protein 3 alpha: Pleiotrophin (PTN); Pleiptrophin: SDF-1β; (CXCL12); Stromal cell derived factor beta: TECK; (CCL25); Thymus-expressed Chemokine: TGF-α; Transforming growth factor alpha: TRANCE (RANK L); (TNFSF11); TNF-related Activin-induced Cytokine: sVAP-1; and Vascular Adhesion Protein-1.

Hormones. Prolactin; Prolactin: IGFBP-2; Insulin-like Growth Factor Binding Protein 2: Leptin/OB; Leptin: Insulin; Insulin: Resistin; Resistin: Adiponectin; Adiponectin: Glucagon; Glucagon: GLP-1; Glucagon-like Peptide 1: and PYY; Peptide YY:

Adiponectins. MCP-1; Monocyte Chemotactic Protein I: Leptin; Leptin: Resistin; Resistin: Adiponectin; Adiponectin: IL-6; IL-6: TNF-α; TNF-alpha: and tPAI-1; thrombin-activatable fibrinolysis inhibitor.

Neuronal Growth Factors. CNTF; Ciliary neurotrophic factor: GDNF; Glial cell line derived neurotrophic factor: BDNF; Brain-derived neurotrophic factor: NT-3; Neurotrophin 3: NT-4; Neurotrophin 4: and β-NGF; beta-Nerve Growth Factor:

Growth Factors. ANG; Angiogenin: EGF; Epidermal growth factor: FGF-7; Fibroblast growth factor-7: FGF-9; Fibroblast growth factor-9: GM-CSF; Granulocyte macrophage colony stimulating factor: GRO-α (MGSA); (CXCL1); Melanoma Growth Stimulating Activity: OSM; Oncostatin M: PIGF; Placenta growth factor: TGF-β3; Transforming growth factor beta-3: AR; Amphiregulin: FGF-6; Fibroblast growth factor-6: G-CSF; Granulocyte colony stimulating factor: SCF; Stem cell factor: VEGF; Vascular endothelial growth factor: CT-1; Cardiotrophin-1: GRO-β; (CXCL2); Growth Related Oncogene beta: HB-EGF; Heparin-Binding EGF-like Growth Factor: HGF; Hepatocyte growth factor: HVEM; (TNFRSF14); Herpesvirus Entry Mediator: MMP-10 (total); Matrix Metalloproteinase 10: MMP-7 (total); Matrix Metalloproteinase 7: MMP-9 (total); Matrix Metalloproteinase 9: TIMP-1; Tissue Inhibitors of Metalloproteinases 1: VEGF-D (FIGF); Vascular Endothelial Growth Factor D: VEGF-R2 (Flk-1/KDR); Vascular Endothelial Growth Factor Receptor 2: FGF basic (FGF-2); Fibroblast growth factor basic: IGF-I; Insulin-like Growth Factor I: IGF-II; Insulin-like Growth Factor II: IGFBP-1; Insulin-like Growth Factor Binding Protein 1: IGFBP-3; Insulin-like Growth Factor Binding Protein 3: IGFBP-4; Insulin-like Growth Factor Binding Protein 4: IGFBP-6; Insulin-like Growth Factor Binding Protein 6: MMP-1 (total); Matrix Metalloproteinase 1: MMP-2 (total); Matrix Metalloproteinase 2: and TIMP-2; Tissue Inhibitors of Metalloproteinases 2.

Soluble Receptors. sCD23; None: Fas; Fas (CD95): IL-1ra; Interleukin 1 receptor antagonist: IL-2 sRα; Interleukin 2 soluble receptor alpha: TRAIL; (TNFSF10); TNF-related apoptosis inducing ligand: uPAR; Urokinase-type plasminogen activator receptor: Flt-3 L; fms-like tyrosine kinase-3 ligand: sgp130; Soluble glycoprotein 130: IL-1 sRI; Interleukin 1 soluble receptor I: IL-6 sR; Interleukin 6 soluble receptor: TNF RI; Tumor necrosis factor receptor I: TNF-RII; Tumor necrosis factor receptor II: sVE-cadherin; Vascular Epithelium Cadherin: CCL28; CCL28: CTLA-4; Cyotoxic T-lymphocyte-associated Molecule 4: DR6; (TNFRSF21); Death Receptor 6: Fas Ligand; (TNFSF6); Fas Ligand: ICAM-3 (CD50); Intercellular adhesion Molecule 3: IL-2 Rγ; Interleukin 2 receptor gamma: IL-5 Ra (CD125); Interleukin 5 receptor alpha: L-Selectin (CD62L); L-selectin: PECAM-1 (CD31); Platelet Endothelial Cell Adhesion Molecule-1: SCF R; Stem Cell Factor Receptor: TRAIL R4; (TNFRSFIOD); TNF-related Apoptosis-inducing Ligand Receptor 4: ALCAM (CD166); Activated Leukocyte Cell Adhesion: CD27; (TNFRSF7); CD27: CD30; (TNFSF8); CD30: CD40; CD40: CNTF Rα; Ciliary neurotrophic factor receptor: ICAM-1 (CD54); Intercellular Adhesion Molecule 1: IGF-I R; Insulin-like Growth Factor I Receptor: IL-1 sRII; Interleukin 1 soluble receptor II: IL-2 Rβ; Interleukin 2 receptor beta: IL-10 Rβ; Interleukin 10 receptor beta: M-CSF R; Macrophage colony stimulating factor receptor: PDGF Rα; Platelet-derived Growth factor Receptor alpha: and TRAIL R4; (TNFRSF10D); TNF-related Apoptosis-inducing Ligand Receptor 4.

In another aspect, the invention provides methods of assessing the health status of an animal by determining the relative activity or expression of a set of analytes. Generally, the methods comprise the steps of:
a) obtaining a biological sample from the animal; where the sample at least putatively contains a predetermined set of analytes of interest. The set of analytes comprises at least one cytokine, chemokine, hormone, and adipokine.
b) contacting the sample with a collection of molecular probes for determining the activity or presence of each of the predetermined set of analytes, wherein for each analyte in the set, the collection of molecular probes comprises at least one probe suitable for detecting the presence or a measurable activity of that analyte. Each probe in the collection is capable of producing an independently detectable signal when the analyte corresponding to that probe is present in the sample.
c) detecting the independently detectable signals produced after the sample is contacted with the collection.
d) correlating the detectable signals with at least the relative presence or activity of each of the predetermined set of analytes in the sample;
e) correlating the relative presence or activity of each of the predetermined set of analytes in the sample with known or determined parameters of health status; and
f) making a determination of the health status of the animals in accordance with the correlations made in step e).

In another embodiment, the set of analytes comprises and set of genes and the method comprises the steps of:
a) obtaining a biological sample from the animal; where the sample at least putatively contains a predetermined set of genes of interest or the expression products of those genes. The set of genes comprises at least one gene each encoding a cytokine, a chemokine, a hormone, and an adipokine.
b) contacting the sample with a collection of molecular probes for determining the activity or expression of each of the predetermined set of genes, wherein for each gene in the set, the collection of molecular probes comprises at least one probe suitable for detecting the activity or expression of that gene. Each probe in the collection is capable of producing an independently detectable signal when the gene or expression product of the gene corresponding to that probe is present in the sample.
c) detecting the independently detectable signals produced after the sample is contacted with the collection.
d) correlating the detectable signals with at least the relative activity or expression of each of the predetermined set of genes in the sample;
e) correlating the relative activity or expression of each of the predetermined set of genes in the sample with known or determined parameters of health status; and
f) making a determination of the health status of the animals in accordance with the correlations made in step e).

The methods have many actual and potential uses. Table 1 shows a partial list of some such uses.

TABLE 1

| Physiological condition to be | Current State of the art | Parameters to be measured with the |
|---|---|---|
| assessed | diagnosis | methods provided |
| Insulin Resistance | Glucose tolerance test | Decrease in adiponectin; |
| | | Increase in insulin, IL-6, and TNF- |
| | | alpha; Alterations in Leptin and |
| | | resistin |
| Predisposition to Type II | Glucose tolerance test to | Decrease in adiponectin; Increase in |
| Diabetes | establish insulin resistance | insulin, IL-6, and TNF-alpha (e.g. to |
| | | document low grade inflammation); |
| | | Alterations in leptin and resistin |
| Dietary intervention, e.g. | None | Analysis of changes in cytokine/ |
| caloric restriction | | chemokine/endocrine molecules can |
| | | be used to molecularly explain |
| | | underlying physiology and e.g. |
| | | develop improved strategies for weight |
| | | loss |
| Inflammation | | Establish improved profiles of both |
| | | pro and anti inflammatory cytokines |
| | | (e.g. upregulation) |
| | | Improved homeostatis in system leads |
| | | to better handling of inflammation or |
| | | immune challenges. |
| Allergies | | Develop surrogate marker based on |
| | | Thelper1 and T helper 2 cytokines to |
| | | predict development of allergies |
| | | and/or efficacy of a dietary |
| | | intervention |
| Aging | | Develop surrogate marker for aging to |
| | | evaluated nutritional regimens that |
| | | delay or prevent aging, or promote |
| | | longevity |

In certain embodiments, the set of analytes further comprises one or more of a neuronal growth factor, a growth factor other than a neuronal growth factor, or a soluble receptor, in addition to the cytokine, chemokine, hormone, and adipokine discussed herein. Where the analytes are genes, the set comprises the corresponding genes, in accordance with the foregoing limitations. Preferably, the detectable probes are specific for detecting a the presence or activity of each of the proteins (or encoded gene product of each of the set of genes), or the activity or expression of each of the genes.

The detectable probes comprise antibodies, antibody fragments, ligands, receptors, or binding proteins, nucleic acids, for example DNA or RNA. Preferably, wherein the set comprises the proteins, the collection of probes comprises antibodies for each of the proteins.

In one embodiment of the methods as discussed herein for the collections of probes, the set of proteins or genes comprises one or more genes encoding, or proteins which are the cytokines interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III.

In various embodiments, the predetermined set of analytes may, alternatively or in addition to the foregoing, include one or more proteins which are or genes encoding the chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1.

In yet other embodiments, the predetermined set of genes or proteins includes one or more genes encoding or proteins which are the hormones prolactin, insulin-like growth factor binding protein 2, leptin, insulin, resistin, adiponectin, glucagon, glucagon-related peptide 1, or PYY.

In one embodiment, the predetermined set of genes or proteins may also include one or more genes encoding or proteins which are the adipokines monocyte chemotactic protein 1, leptin, resistin, adiponectin, IL-6, TNF-alpha, or thrombin-activatable fibrinolysis inhibitor.

In other embodiments, the predetermined set of genes or proteins further variously comprises one or more genes encoding one or more of the following, or the proteins themselves: the neuronal growth factors ciliary neurotrophic factor, glial cell line derived neurotrophic factor, brain-derived neurotrophic factor, neurotrophin 3, neurotrophin 4, or beta-nerve growth factor; the growth factors angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2; or the soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cyotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4.

In one preferred embodiment of the method, the predetermined set of analytes comprises one or more of genes encoding each, of IL-2, IL-4, IL-6, IL-7, IL-8, IL-10, IL-18, IFN γ, IP-10, TNF-α, MCP-1, GLP-1, glucagon, insulin, adiponectin, and resistin. In another embodiment, the set of analytes comprises the foregoing proteins themselves. In various embodiments, the animal is human, murine, simian, canine, or feline. The predetermined set of analytes further comprises one or more genes encoding IL-15, KC, or leptin, or the proteins themselves, in certain embodiments.

In one preferred embodiment of the method, the analytes are from a canine and the probes are antibodies specific for the proteins or expression products of the genes encoding them. In one embodiment, the collection of molecular probes allow for a quantitative determination of the amount of the protein, or the expression of each gene.

The method can be practiced with each probe attached to a matrix, wherein each such attached probe remains competent to provide a quantitative determination of the amount of expression of a gene corresponding to that probe, in a sample brought into contact with the matrix.

In some embodiments, the method further comprises a step of contacting the sample and the collection of molecular probes with a set of secondary antibodies comprising one or more antibodies that can, for example, detect the presence of a particular portion or type of an antibody, detect specific binding between an expression product of each gene in the set, and a corresponding probe in the collection. The use of secondary antibodies for such detection is understood by those of skill in developing antibody-based assays, such as certain ELISA methods and various so-called “sandwich” techniques. Either the probe or a second antibody can be further linked to a signal generation or amplification system, such as an enzyme.

The methods in certain embodiments involve the use of a collection of molecular probes wherein each probe is attached to a separate matrix, such as a bead or a polymeric support material.

The methods involve the use of a biological sample, which is preferably a sample which is likely to contain the genes in the predetermined set, or is likely to contain the expression products thereof, is easy and relatively painless to obtain, is abundant or whose absence will do the animal no harm, and is reproducible. Various such biological samples will occur to those of skill, examples includes samples of various tissues and fluids. Examples include blood, serum, plasma, urine, tissue extracts, cerebral spinal fluid (CFS), synovial fluid, and cellular extracts. Tissue culture cells, extracts, supernatant fluids, and spent culture medium are also useful herein. Preferred samples are blood, serum, and plasma. Sample size is not critical, samples can be of any size that is useful, practical, and analytically meaningful. Samples of less than 1 ml are preferred. Samples typically are less than 100 μl for example 75 or 50 μl. Smaller samples are also useful herein. Sample that are sufficiently small to allow assays in standard laboratory equipment, are of course preferred, as are miniaturized assays.

In another aspect, the invention provides methods of formulating a nutritional regimen for improving the health of an animal. The methods comprise
a) selecting a predetermined set of analytes of interest in an animal wherein the activity of the analytes, or presence thereof can be correlated with the health status of the animal, and with a nutritional regimen of the animal, the set comprising at least one protein or one gene encoding each of a cytokine, a chemokine, a hormone, and an adipokine;
b) obtaining a biological sample from the animal; said sample putatively containing the predetermined set of analytes, or the expression products thereof, said sample indicative of a current nutritional regimen of the animal;
c) determining a baseline measurement by contacting the sample with a collection of molecular probes for determining the activity or presence of each of the predetermined set of analytes, or expression product thereof, wherein for each analyte in the set, the collection of molecular probes comprises at least one probe suitable for detecting the activity or presence of that analyte, each probe capable of producing an independently detectable signal when the analyte, or the expression product of the analyte corresponding to that probe is present in the sample;
d) detecting the independently detectable signals produced after the sample is contacted with the collection,
e) correlating the detectable signals with the relative activity or presence of each of the predetermined set of analytes, or expression product thereof, in the sample;
f) correlating the relative activity or expression of each of the predetermined set of genes in the sample with known parameters of health status;
g) making a determination of the health status of the animal on the current nutritional regimen in accordance therewith;
h) formulating one or more test nutritional regimens or supplements for testing in the animal by adjusting one or more of the macronutrient content or source, the micronutrient content or source, supplemental dietary components, or caloric content of the diet as compared to the current nutritional regimen of the animal;
i) providing one or more test nutritional regimens or supplements, or combination thereof, to the animal in an amount and for a time effective to change the activity, presence or expression of one or more of the predetermined set of analytes;
j) for each test nutritional regimen or supplement, or combination thereof, repeating steps b) through g) with a new sample from the animal to determine whether the test nutritional regimen or supplement, or combination thereof has improved the health status of the animal; and
k) selecting a formulated nutritional regimen or supplement, or combination thereof, which improves the heath status of the animal.

The skilled artisan will appreciate that step j) refers to repeating steps b) through g) however these steps are repeated with a new sample taken from the animal after step j), i.e., after the animal has received the new nutritional regimen.

As with the other methods and compositions provided herein, the selection of the predetermined analytes is essentially a rational process of selecting those analytes, the presence, activity, or expression of which can be correlated with both health status and a nutritional regimen, and which will provide useful results. Selection of analytes that have zero correlation with health status and zero correlation any nutritional regimen is to be avoided, although such analytes may be included as controls or test analytes, or the like.

The methods described herein are useful for the formulation of any nutritional regimen with any animal as described herein. In various preferred embodiments, the methods are quite useful in situations wherein the animal is obese, has diabetes, has symptoms of being predisposed to diabetes, has an undesirable level of inflammation, has an undesirable level of insulin resistance, has metabolic syndrome, premature atherosclerosis, abnormal glucose metabolism, or abnormal fat metabolism. Many such conditions are known in the art and can be loosely or more strictly associated with nutritional regimens, particularly long-term nutritional regimens. In one embodiment, the formulated nutritional regimen, supplement, or combination thereof improves the immune function, reduces inflammation, reduces insulin resistance, or a combination thereof in the animal.

In various embodiments, the formulated nutritional regimen, supplement, or combination thereof has one or more of the following effects (1) reduces inflammation in the animal, and one or more of increases anti-inflammatory cytokines, reduces pro-inflammatory cytokines, or decreases cytokine mediators of inflammation; (2) reduces insulin resistance in the animal and one or more of increases adiponectin, decreases resistin, or decreases leptin; or (3) reduces one or more of dyslipidemia, inflammation, hypertension, altered vascular reactivity, or visceral obesity, or improves fibrinolysis. In specific embodiments, the set of analytes comprise each and any of the analytes described herein for other aspects of the invention.

In a preferred embodiment, the predetermined set of analytes comprises one or more genes encoding each of IL-2, IL-4, IL-6, IL-7, IL-8, IL-10, IL-18, IFN γ, IP-10, TNF-α, MCP-1, GLP-1, glucagon, insulin, adiponectin, and resistin, or the corresponding proteins. Additionally, one or more genes encoding IL-15, KC, or leptin may be included, or the corresponding proteins.

In certain embodiments, the animal is human, murine, simian, canine, or feline. In a preferred embodiment, the analytes are proteins from a canine and the molecular probes are antibodies that allow for a quantitative determination of the amount of such proteins. In some embodiments, each probe is attached to a matrix and remains capable of providing a quantitative determination of the amount of the analyte corresponding to that probe when a sample contacts the matrix.

The methods can also comprise the further step of contacting the sample and the collection of molecular probes with a set of secondary probes, e.g., secondary antibodies comprising one or more antibodies that increase specificity or increase signal through amplification. In one embodiment, each probe is attached to a separate matrix.

As described herein, the sample can be any biological sample such as tissue or bodily fluid. Examples include blood, serum, plasma, urine, tissue extracts, cerebral spinal fluid (CFS), synovial fluid, and cellular extracts. Ex vivo samples such as stably- or transiently-cultured tissue or cells, or supernatant fluid, spent culture medium, exudates thereof, or the like, are also useful herein. Samples comprising or consisting of serum or plasma are preferred for use in the methods.

In a further aspect, the present invention provides kits suitable for determining in a single sample, the activity, presence, or expression of each of a predetermined set of analytes. The kits comprise in separate containers in a single package or in separate containers in a virtual package, as appropriate for the kit component, a multiplex assay comprising a collection of detectable molecular probes as defined herein and one or more of (1) instructions for how to use the multiplex assay to determine the activity, presence, or expression of each of a predetermined set of analytes, (2) instruction for how to assess the health status of an animal using the multiplex assay, (3) instructions for formulating a nutritional regimen for improving the health of an animal using the multiplex assay, and (4) one or more ingredients suitable for consumption by an animal. In certain embodiments, the kit comprises the multiplex assay and a food composition such as a nutritionally complete food for pets or nutritional supplements such as vitamins and minerals that are useful for formulating a nutritional regimen for improving the health of an animal.

When the kit comprises a virtual package, the kit is limited to instructions in a virtual environment in combination with one or more physical kit components. The kit may contain additional items such as a device for mixing reagents useful with the multiplex assay or a device for supporting and/or handling the multiplex assay.

In another aspect, the present invention provides a means for communicating information about or instructions for using the multiplex assay for one or more of (1) determining in a single sample, the activity, presence, or expression of each of a predetermined set of analytes, (2) assessing the health status of an animal, or (3) formulating a nutritional regimen for improving the health of an animal. The means comprises a document, digital storage media, optical storage media, audio presentation, or visual display containing the information or instructions. In certain embodiments, the communication means is a displayed web site, visual display kiosk, brochure, product label, package insert, advertisement, handout, public announcement, audiotape, videotape, DVD, CD-ROM, computer readable chip, computer readable card, computer readable disk, computer memory, or combination thereof containing such information or instructions. Useful information includes one or more of (1) methods and techniques for handling biological samples for use with the multiplex assay (2) contact information for Individuals to use if they have a question about the multiplex assay and its use.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a cytokine” includes a plurality of such cytokines. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

### EXAMPLES

The invention can be further illustrated by the following examples, although it will be understood that these examples are included merely for purposes of illustration and are not intended to limit the scope of the invention unless otherwise specifically indicated.

### Example 1

Mouse Study with Modified Dietary Regimen Comprising Fish Oil, and Vitamin E. The objective of the experiment was to identify molecular pathways influenced by modified diet and understand mechanisms of action with regard to increased life span. Experimental Design: Group I: Mice fed Modified diet from 10-16 months [n=19]; Group II: Mice fed Control diet from 10-16 months [n=19]; Group III: Mice fed Modified diet from 17-23 months [n=7]; and Group IV: Mice fed Control diet from 17-23 months [n=9]. Experiment: Use multiplex method to measure levels of 27 proteins (MIP-1α, GM-CSF, MCP-1, KC, RANTES, IFN-γ, IL-1β, IL-1α, G-CSF, IP-10, IL-2, IL-4, IL-5, IL-6, IL-7, IL-10, IL-12, TNF-α, IL-9, IL-13, IL-15, IL-17, Insulin, Leptin, tPAI-1, Resistin, and Adiponectin) using 75 μl plasma samples. Multiplex measurements taken with panel of probes corresponding to the 27 selected proteins, using Luminex device. Timepoints: Male C57B1/6 mice were used. Mice were started on the dietary regimens at either 10 or 17 months of age. Mice were fed either modified (“test”) diet or control diet for a period of 6 months—from either 10 to 16 months of age (“Y”, young mice, Groups 1 and II), or from 17 to 23 months of age (“O”, old mice, Groups III and IV). Blood samples were obtained form euthanized mice and the organs were flash-frozen to generate a tissue bank. The biosamples were analyzed for the 27 protein analytes. The results are shown in Table 2.

TABLE 2A
Cytokines

| Sample | | | | | | | | |
|---|---|---|---|---|---|---|---|---|
| Identification | | MIP-1α | GM-CSF | MCP-1 | KC | RANTES | IFN-γ | IL-1β |
| (Age - months) | | pg/ml | pg/ml | pg/ml | pg/ml | pg/ml | pg/ml | pg/ml |
| C1-12 | 10 | 83 | 37 | 18 | 35 | 8 | 12 | <3 |
| C2-12 | 10 | 147 | 67 | 40 | 32 | 13 | 14 | 3 |
| C3-12 | 10 | 99 | 7 | 29 | 24 | 16 | 32 | 11 |
| C4-12 | 10 | 64 | 35 | 23 | 31 | 12 | 6 | 4 |
| C5-12 | 10 | 632 | 216 | 47 | 77 | 40 | 8 | 14 |
| C6-12 | 10 | 110 | 69 | 9 | 80 | 38 | <3 | <3 |
| C7-12 | 10 | 4 | 13 | 26 | 34 | 26 | <3 | 13 |
| C8-12 | 10 | 94 | 25 | 25 | 134 | 23 | <3 | <3 |
| C9-12 | 10 | 88 | 48 | 19 | 21 | 30 | 2,187 | <3 |
| C10-12 | 10 | 407 | 151 | 21 | 33 | 30 | <3 | <3 |
| C11-12 | 10 | 679 | 117 | 29 | 128 | 6 | 51 | 16 |
| C12-12 | 10 | 105 | 25 | 13 | 18 | 34 | <3 | <3 |
| C-13 | 10 | 125 | 31 | 21 | 8 | 48 | <3 | <3 |
| C14-12 | 10 | 105 | <5 | 9 | 95 | 26 | <3 | <3 |
| C15-12 | 10 | 338 | 90 | 18 | 27 | 21 | <3 | 5 |
| C16-12 | 10 | 99 | 33 | <6 | 107 | 13 | <3 | <3 |
| C17-12 | 10 | 83 | 45 | 16 | 51 | 31 | <3 | <3 |
| C18-12 | 10 | 210 | 110 | 29 | 419 | 25 | <3 | 5 |
| C19-12 | 10 | 176 | 47 | 13 | 59 | 17 | <3 | <3 |
| C20-12 | 10 | 4,861 | 52 | 26 | 16 | 20 | 12 | 7 |
| T1-12 | 10 | 4 | 23 | <6 | 12 | 14 | <3 | <3 |
| T2-12 | 10 | 77 | 101 | <6 | 27 | 17 | <3 | <3 |
| T3-12 | 10 | 241 | 77 | 24 | 45 | 38 | 13 | <3 |
| T4-12 | 10 | 99 | 65 | 162 | 71 | 43 | <3 | 6 |
| T5-12 | 10 | 115 | 52 | 11 | 40 | 20 | <3 | <3 |
| T6-12 | 10 | 105 | 71 | 19 | 30 | 36 | <3 | <3 |
| T7-12 | 10 | 120 | 156 | 32 | 79 | 52 | <3 | <3 |
| T8-12 | 10 | 94 | <5 | 11 | 5 | 34 | 4 | <3 |
| T9-12 | 10 | 210 | 187 | 25 | 67 | 50 | <3 | 10 |
| T10-12 | 10 | 147 | 180 | 89 | 59 | 42 | 9 | 83 |
| T11-12 | 10 | 414 | 123 | 24 | 70 | 25 | 5 | 8 |
| T12-12 | 10 | 70 | 38 | 6 | 30 | 10 | <3 | <3 |
| T13-12 | 10 | 41 | 63 | 15 | <3 | 43 | <3 | <3 |
| T14-12 | 10 | 70 | <5 | 13 | 20 | 4 | 5 | <3 |
| T15-12 | 10 | 267 | 72 | 90 | 47 | 25 | <3 | 14 |
| T16-12 | 10 | 88 | 63 | 15 | 26 | 22 | <3 | <3 |
| T17-12 | 10 | 41 | 35 | <6 | 94 | 18 | <3 | <3 |
| T19-12 | 10 | 180 | 34 | 23 | 59 | 31 | <3 | <3 |
| T20-12 | 10 | 120 | 52 | 34 | 63 | 18 | <3 | 5 |
| T1-12 | 20 | <3 | <5 | <6 | 17 | <3 | <3 | <3 |
| T3-12 | 20 | 301 | 55 | 34 | 20 | 21 | 4 | 9 |
| T5-12 | 20 | 650 | 107 | 49 | 57 | 14 | 41 | 83 |
| T6-12 | 20 | 160 | 15 | 18 | 29 | 9 | 35 | <3 |
| T7-12 | 20 | <3 | <5 | 15 | <3 | 11 | <3 | 3 |
| T8-12 | 20 | 228 | 76 | 51 | 19 | 20 | 229 | 19 |
| T12-12 | 20 | 192 | 57 | 30 | 110 | 20 | 4 | 5 |
| T16-12 | 20 | 1,500 | 262 | 102 | 213 | 17 | 144 | 44 |
| T17-12 | 20 | 312 | 103 | 1,562 | 74 | 452 | 51 | 1,175 |
| C1-12 | 20 | 419 | 69 | 15 | 40 | 14 | 47 | <3 |
| C2-12 | 20 | 286 | 59 | 56 | 33 | 24 | 87 | 25 |
| C3-12 | 20 | 147 | 88 | 304 | 64 | 124 | 54 | 423 |
| C5-12 | 20 | 365 | <5 | 1,426 | 9 | 645 | 4 | 444 |
| C6-12 | 20 | 88 | 48 | 44 | 27 | 13 | <3 | 11 |
| C9-12 | 20 | 389 | 65 | 461 | 110 | 64 | 31 | 614 |
| C10-12 | 20 | 143 | 122 | 41 | 39 | 18 | <3 | 3 |

| Sample | | | | | | | | |
|---|---|---|---|---|---|---|---|---|
| Identification | | IL-1α | G-CSF | IP-10 | IL-2 | IL-4 | IL-5 | IL-6 |
| (Age - months) | | pg/ml | pg/ml | pg/ml | pg/ml | pg/ml | pg/ml | pg/ml |
| C1-12 | 10 | 20 | 36 | 35 | 3 | <3 | 3 | <3 |
| C2-12 | 10 | 11 | 26 | 34 | <3 | <3 | 7 | 7 |
| C3-12 | 10 | 17 | 105 | 371 | <3 | <3 | 8 | 6 |
| C4-12 | 10 | 33 | 136 | 64 | <3 | <3 | 8 | 10 |
| C5-12 | 10 | 51 | 274 | 86 | 21 | 4 | 30 | 35 |
| C6-12 | 10 | 24 | 102 | 71 | 9 | <3 | 28 | 2 |
| C7-12 | 10 | 10 | 36 | 45 | 42 | <3 | 11 | 7 |
| C8-12 | 10 | 81 | 463 | 80 | <3 | <3 | 17 | 122 |
| C9-12 | 10 | 26 | 87 | 93 | <3 | <3 | 5 | 7 |
| C10-12 | 10 | 16 | 168 | 74 | 4 | 7 | 9 | 5 |
| C11-12 | 10 | 22 | 93 | 117 | <3 | <3 | 7 | 6 |
| C12-12 | 10 | 14 | 65 | 29 | <3 | <3 | 15 | 5 |
| C-13 | 10 | 37 | 80 | 44 | <3 | <3 | 26 | 7 |
| C14-12 | 10 | 20 | 141 | 30 | <3 | <3 | 6 | 18 |
| C15-12 | 10 | 21 | 83 | 38 | 4 | <3 | 11 | 5 |
| C16-12 | 10 | 23 | 76 | 56 | <3 | <3 | 5 | 3 |
| C17-12 | 10 | 22 | 100 | 46 | 19 | <3 | 29 | 8 |
| C18-12 | 10 | 241 | 2,328 | 42 | 10 | <3 | 22 | 317 |
| C19-12 | 10 | 20 | 42 | 32 | 4 | <3 | 8 | <3 |
| C20-12 | 10 | 9 | 24 | 26 | 5 | <3 | 7 | 6 |
| T1-12 | 10 | 8 | 57 | 6 | 4 | <3 | 4 | <3 |
| T2-12 | 10 | 14 | 202 | 46 | 6 | <3 | 8 | <3 |
| T3-12 | 10 | 34 | 160 | 73 | 3 | <3 | 31 | 8 |
| T4-12 | 10 | 31 | 193 | 712 | <3 | <3 | 53 | 3 |
| T5-12 | 10 | 12 | 30 | 36 | 7 | <3 | 13 | <3 |
| T6-12 | 10 | 21 | 92 | 53 | 5 | <3 | 10 | <3 |
| T7-12 | 10 | 33 | 246 | 104 | 28 | <3 | 14 | <3 |
| T8-12 | 10 | 18 | 83 | 57 | <3 | <3 | 1,092 | <3 |
| T9-12 | 10 | 39 | 224 | 93 | 39 | <3 | 32 | 3 |
| T10-12 | 10 | 38 | 259 | 57 | 10 | <3 | 65 | 75 |
| T11-12 | 10 | 25 | 218 | 41 | 13 | <3 | 19 | <3 |
| T12-12 | 10 | 11 | 53 | 23 | <3 | <3 | 14 | 5 |
| T13-12 | 10 | 19 | 93 | 60 | <3 | <3 | 13 | <3 |
| T14-12 | 10 | 19 | 105 | 32 | <3 | <3 | 4 | <3 |
| T15-12 | 10 | 19 | 62 | 47 | 8 | <3 | 9 | 4 |
| T16-12 | 10 | 35 | 56 | 35 | 6 | <3 | 10 | <3 |
| T17-12 | 10 | 15 | 70 | 38 | <3 | <3 | 6 | <3 |
| T19-12 | 10 | 38 | 87 | 47 | 5 | <3 | 8 | <3 |
| T20-12 | 10 | 68 | 141 | 44 | 4 | <3 | 8 | 26 |
| T1-12 | 20 | 190 | 171 | <6 | <3 | <3 | 8 | 5 |
| T3-12 | 20 | 186 | 879 | 42 | 7 | <3 | 8 | 131 |
| T5-12 | 20 | 42 | 298 | 29 | 11 | <3 | 16 | 13 |
| T6-12 | 20 | 35 | 102 | 16 | <3 | <3 | 8 | <3 |
| T7-12 | 20 | 28 | 85 | 6 | <3 | <3 | 6 | <3 |
| T8-12 | 20 | 99 | 550 | 26 | 3 | <3 | 16 | 47 |
| T12-12 | 20 | 63 | 112 | 9 | 4 | <3 | 12 | 24 |
| T16-12 | 20 | 63 | 115 | 38 | 68 | <3 | 25 | 18 |
| T17-12 | 20 | 79 | 1,818 | 88 | <3 | <3 | 273 | 1,528 |
| C1-12 | 20 | 90 | 698 | 36 | 5 | <3 | 10 | 365 |
| C2-12 | 20 | 150 | 629 | 39 | 10 | <3 | 10 | 44 |
| C3-12 | 20 | 40 | 191 | 433 | <3 | <3 | 175 | 761 |
| C5-12 | 20 | 78 | 396 | 28 | <3 | <3 | 122 | 35 |
| C6-12 | 20 | 83 | 183 | 42 | <3 | <3 | 12 | <3 |
| C9-12 | 20 | 40 | 527 | 104 | 5 | <3 | 83 | 143 |
| C10-12 | 20 | 181 | 199 | 15 | 17 | <3 | 24 | 13 |

| Sample | | | | | | | | | |
|---|---|---|---|---|---|---|---|---|---|
| Identification | | IL-7 | IL-10 | IL-12 | TNFα | IL-9 | IL-13 | IL-15 | IL-17 |
| (Age - months) | | pg/ml | pg/ml | pg/ml | pg/ml | pg/ml | pg/ml | pg/ml | pg/ml |
| C1-12 | 10 | 9 | <10 | <4 | 8 | * | 6 | <9 | 9 |
| C2-12 | 10 | 80 | <10 | 77 | 15 | * | 9 | 11 | 27 |
| C3-12 | 10 | <4 | <10 | 87 | 9 | * | <5 | 11 | 13 |
| C4-12 | 10 | 6 | <10 | <4 | 7 | * | 20 | 18 | 11 |
| C5-12 | 10 | 4 | <10 | 232 | 13 | * | 21 | 56 | 17 |
| C6-12 | 10 | <4 | <10 | 52 | 5 | * | 13 | <9 | 11 |
| C7-12 | 10 | 15 | <10 | 71 | 6 | * | 21 | <9 | 30 |
| C8-12 | 10 | <4 | <10 | 32 | 16 | * | 12 | 220 | 10 |
| C9-12 | 10 | 55 | 72 | 238 | 15 | * | 12 | 17 | 57 |
| C10-12 | 10 | <4 | <10 | 146 | 4 | * | 20 | 22 | 15 |
| C11-12 | 10 | <4 | <10 | 32 | <3 | * | 13 | 13 | 10 |
| C12-12 | 10 | <4 | <10 | 59 | 5 | <11 | 9 | <9 | 13 |
| C-13 | 10 | <4 | <10 | 55 | 5 | * | <5 | 21 | 34 |
| C14-12 | 10 | <4 | <10 | 228 | 8 | <11 | 22 | <9 | 7 |
| C15-12 | 10 | 9 | <10 | 170 | 6 | <11 | 25 | 61 | 7 |
| C16-12 | 10 | <4 | <10 | 69 | <3 | * | 24 | <9 | 6 |
| C17-12 | 10 | <4 | <10 | 170 | 5 | 31 | 19 | 26 | 19 |
| C18-12 | 10 | 9 | <10 | 148 | 18 | * | 31 | 66 | 20 |
| C19-12 | 10 | <4 | <10 | 54 | 4 | <11 | 20 | <9 | 5 |
| C20-12 | 10 | 8 | <10 | 208 | 8 | <11 | 20 | 124 | 11 |
| T1-12 | 10 | <4 | <10 | 47 | 3 | * | 8 | <9 | <3 |
| T2-12 | 10 | <4 | <10 | 35 | 5 | <11 | 7 | <9 | 7 |
| T3-12 | 10 | <4 | <10 | 141 | 7 | <11 | 20 | <9 | 13 |
| T4-12 | 10 | <4 | 20 | 564 | 296 | <11 | 23 | 10 | 19 |
| T5-12 | 10 | <4 | <10 | 25 | 5 | * | 5 | <9 | 7 |
| T6-12 | 10 | <4 | <10 | 70 | 4 | * | 23 | 16 | 4 |
| T7-12 | 10 | 5 | 31 | 285 | 10 | 14 | 24 | 28 | 21 |
| T8-12 | 10 | 6 | <10 | <4 | 4 | * | <5 | 13 | 8 |
| T9-12 | 10 | <4 | <10 | 363 | 9 | * | 20 | 20 | 13 |
| T10-12 | 10 | <4 | 12 | 369 | 60 | * | 10 | <9 | 67 |
| T11-12 | 10 | <4 | <10 | 69 | 20 | * | 26 | 131 | 8 |
| T12-12 | 10 | <4 | <10 | 107 | <3 | * | 20 | <9 | 5 |
| T13-12 | 10 | <4 | <10 | 54 | 9 | * | 11 | 14 | 17 |
| T14-12 | 10 | <4 | <10 | 38 | 3 | * | 13 | <9 | 6 |
| T15-12 | 10 | <4 | <10 | 75 | 27 | 14 | 18 | 10 | 9 |
| T16-12 | 10 | <4 | <10 | 68 | 6 | <11 | 22 | <9 | 6 |
| T17-12 | 10 | <4 | <10 | <4 | 8 | * | 28 | <9 | 9 |
| T19-12 | 10 | <4 | <10 | 38 | 6 | 19 | 24 | <9 | 8 |
| T20-12 | 10 | <4 | <10 | 52 | 14 | <11 | 24 | 29 | 25 |
| T1-12 | 20 | <4 | <10 | 22 | 9 | <11 | <5 | <9 | 6 |
| T3-12 | 20 | <4 | 20 | 105 | 20 | * | 28 | 16 | 10 |
| T5-12 | 20 | <4 | <10 | 128 | 7 | 103 | 39 | 45 | 14 |
| T6-12 | 20 | 6 | <10 | 12 | 5 | * | <5 | 29 | 4 |
| T7-12 | 20 | <4 | <10 | 35 | 4 | * | 13 | <9 | 6 |
| T8-12 | 20 | 7 | 43 | 190 | 15 | * | 24 | <9 | 61 |
| T12-12 | 20 | <4 | <10 | 85 | 11 | * | 13 | 39 | 8 |
| T16-12 | 20 | <4 | <10 | 135 | 44 | 658 | 31 | 21 | 34 |
| T17-12 | 20 | 5 | 261 | 2,543 | 1,233 | * | 35 | 14 | 1,126 |
| C1-12 | 20 | <4 | 15 | 153 | 11 | 169 | 16 | 51 | 128 |
| C2-12 | 20 | 10 | 48 | 175 | 23 | 187 | <5 | 20 | 43 |
| C3-12 | 20 | 274 | 29 | 999 | 111 | * | <5 | 449 | 276 |
| C5-12 | 20 | 11 | 92 | 2,628 | >10,000 | <11 | <5 | 32 | 841 |
| C6-12 | 20 | 200 | <10 | 103 | 8 | <11 | 16 | 160 | 15 |
| C9-12 | 20 | <4 | 33 | 208 | 91 | * | 11 | 27 | 183 |
| C10-12 | 20 | <4 | <10 | 141 | 7 | * | 12 | 56 | 11 |

TABLE 2B
Adipokines

| Sample | | | | | |
|---|---|---|---|---|---|
| Identification | | Insulin | Leptin | tPAI-1 | Resistin |
| C1-12 | 10 month | 840 | 6,229 | 1,486 | 3,334 |
| C2-12 | 10 month | 906 | 9,992 | 836 | 807 |
| C3-12 | 10 month | 728 | 11,529 | 732 | 727 |
| C4-12 | 10 month | 474 | 12,431 | 398 | 1,360 |
| C5-12 | 10 month | 618 | 4,611 | 2,753 | 2,127 |
| C6-12 | 10 month | 1,361 | 9,748 | 1,609 | 955 |
| C7-12 | 10 month | 939 | 13,068 | 653 | 978 |
| C8-12 | 10 month | 462 | 6,728 | 1,794 | 484 |
| C9-12 | 10 month | 599 | 14,251 | 2,464 | 999 |
| C10-12 | 10 month | 971 | 13,112 | 1,452 | 1,107 |
| C11-12 | 10 month | 515 | 8,270 | 216 | 582 |
| C12-12 | 10 month | 660 | 9,170 | 2,075 | 815 |
| C-13 | 10 month | 564 | 13,499 | 2,378 | 1,035 |
| C14-12 | 10 month | 1,213 | 11,812 | 1,954 | 736 |
| C15-12 | 10 month | 1,116 | 6,027 | 503 | 1,149 |
| C16-12 | 10 month | 632 | 6,772 | 2,289 | 680 |
| C17-12 | 10 month | 574 | 11,722 | 2,927 | 1,565 |
| C18-12 | 10 month | 316 | 813 | 3,880 | 714 |
| C19-12 | 10 month | 686 | 2,170 | 1,244 | 266 |
| C20-12 | 10 month | 761 | 6,536 | 706 | 668 |
| T1-12 | 10 month | 1,158 | 4,674 | 1,047 | 498 |
| T2-12 | 10 month | 836 | 5,872 | 743 | 538 |
| T3-12 | 10 month | 1,226 | 3,919 | 1,170 | 690 |
| T4-12 | 10 month | 728 | 6,397 | 2,342 | 349 |
| T5-12 | 10 month | 840 | 3,402 | 843 | 830 |
| T6-12 | 10 month | 733 | 15,217 | 2,002 | 1,057 |
| T7-12 | 10 month | 1,417 | 12,485 | 1,521 | 1,261 |
| T8-12 | 10 month | 896 | 8,986 | 2,321 | 683 |
| T9-12 | 10 month | 843 | 6,250 | 1,758 | 313 |
| T10-12 | 10 month | 298 | 5,421 | 1,458 | 763 |
| T11-12 | 10 month | 916 | 7,344 | 329 | 330 |
| T12-12 | 10 month | 449 | 3,687 | 1,702 | 123 |
| T13-12 | 10 month | 776 | 6,292 | 2,266 | 1,599 |
| T14-12 | 10 month | 430 | 6,428 | 1,894 | 970 |
| T15-12 | 10 month | 574 | 2,455 | 938 | 405 |
| T16-12 | 10 month | 1,085 | 2,852 | 478 | 351 |
| T17-12 | 10 month | 909 | 6,972 | 1,771 | 259 |
| T19-12 | 10 month | 646 | 3,716 | 1,229 | 430 |
| T20-12 | 10 month | 307 | 2,017 | <12 | 300 |
| T1-12 | 20 month | 847 | 4,785 | 1,839 | 315 |
| T3-12 | 20 month | 492 | 135 | 2,457 | 613 |
| T5-12 | 20 month | 449 | 2,736 | 369 | 425 |
| T6-12 | 20 month | 728 | 2,177 | 216 | 218 |
| T7-12 | 20 month | 564 | 1,738 | 940 | 343 |
| T8-12 | 20 month | 278 | 67 | 2,065 | 613 |
| T12-12 | 20 month | 443 | 1,494 | 249 | 340 |
| T16-12 | 20 month | 402 | 1,013 | 778 | 432 |
| T17-12 | 20 month | 456 | 2,143 | 1,206 | 1,043 |
| C1-12 | 20 month | 258 | 1,688 | 1,049 | 513 |
| C2-12 | 20 month | 258 | 2,663 | 2,400 | 521 |
| C3-12 | 20 month | 341 | 9,421 | 1,419 | 624 |
| C5-12 | 20 month | 803 | 4,347 | 1,293 | 516 |
| C6-12 | 20 month | 919 | 4,431 | 1,835 | 335 |
| C9-12 | 20 month | 521 | 5,676 | 1,517 | 340 |
| C10-12 | 20 month | 521 | 291 | 1,210 | 210 |

TABLE 2C
Adiponectin

| Sample Identification | | Mouse Adiponectin |
|---|---|---|
| C1-12 | 10 month | 8.6 |
| C2-12 | 10 month | QNS |
| C3-12 | 10 month | 13.2 |
| C4-12 | 10 month | 13.0 |
| C5-12 | 10 month | 11.3 |
| C6-12 | 10 month | 10.4 |
| C7-12 | 10 month | 13.4 |
| C8-12 | 10 month | 7.4 |
| C9-12 | 10 month | 10.7 |
| C10-12 | 10 month | 10.0 |
| C11-12 | 10 month | 9.3 |
| C12-12 | 10 month | 11.9 |
| C-13 | 10 month | 10.3 |
| C14-12 | 10 month | 13.3 |
| C15-12 | 10 month | 13.0 |
| C16-12 | 10 month | 8.3 |
| C17-12 | 10 month | 12.7 |
| C18-12 | 10 month | 5.3 |
| C19-12 | 10 month | 9.4 |
| C20-12 | 10 month | 13.8 |
| T1-12 | 10 month | 13.2 |
| T2-12 | 10 month | 8.4 |
| T3-12 | 10 month | 11.7 |
| T4-12 | 10 month | 8.9 |
| T5-12 | 10 month | 11.4 |
| T6-12 | 10 month | 9.1 |
| T7-12 | 10 month | 7.3 |
| T8-12 | 10 month | 8.6 |
| T9-12 | 10 month | 9.3 |
| T10-12 | 10 month | 7.7 |
| T11-12 | 10 month | 10.0 |
| T12-12 | 10 month | 14.7 |
| T13-12 | 10 month | 10.3 |
| T14-12 | 10 month | 14.1 |
| T15-12 | 10 month | 9.2 |
| T16-12 | 10 month | 12.9 |
| T17-12 | 10 month | 12.7 |
| T19-12 | 10 month | 11.8 |
| T20-12 | 10 month | 7.7 |
| T1-12 | 20 month | 16.2 |
| T3-12 | 20 month | 9.1 |
| T5-12 | 20 month | 13.9 |
| T6-12 | 20 month | 19.1 |
| T7-12 | 20 month | 17.7 |
| T8-12 | 20 month | 9.6 |
| T12-12 | 20 month | 13.1 |
| T16-12 | 20 month | 19.3 |
| T17-12 | 20 month | 14.3 |
| C1-12 | 20 month | 8.7 |
| C2-12 | 20 month | 8.0 |
| C3-12 | 20 month | 10.3 |
| C5-12 | 20 month | 9.6 |
| C6-12 | 20 month | 19.5 |
| C9-12 | 20 month | 13.3 |
| C10-12 | 20 month | 10.8 |

Referring to Tables 2A, 2B, and 2C, the data show that a modified dietary regimen reduced inflammation [p≦0.05] in the mice. Pro-inflammatory cytokines IL-6 [Y] & IL-12 [O] decreased; inflammatory mediators IL-7 [Y,O], IL-15 [O] & IL-17 [O] decreased; anti-inflammatory cytokines IL-10 [O] & IL-13 [O] increased. ‘O’ refers to significant in old mice only, ‘Y’ refers to significant in young mice group only and ‘OY’ refers to significant in both young and old mice. Phenotypically, the older mice in the test group had very few incidences of atopic dermatitis as compared to the control groups indicating a reduction in the inflammatory process that are typical of the aging process. Pro-inflammatory cytokines IL-6 and IL-12 decreased in Groups I and III respectively. Cytokines considered inflammatory mediators (IL-7, IL-15, & IL-17) decreased. IL-7 decreased in Groups I and III, while IL-15 and IL-17 decreased in Group III. The anti-inflammatory cytokines IL-10 and IL-13 increased in Group III, consistent with the above observation on inflammation. The modified nutritional regimen reversed insulin resistance [p≦0.05]. In Group III mice an increase in adiponectin was observed. In Group I mice, resistin and leptin were each observed to be lowered. Clearly, the test diet reduced inflammation and reversed insulin resistance, both of which are associated with characteristics typical of old age. The data also shows that various biomarkers can be used to assay the health status of animals.

### Example 2

### Multiplex Assay Development and Optimization

Materials & Methods: Multiplexed assays were developed in three steps: [1] Reagent Procurement/Characterization: Antibodies specific to the canine molecules for the Canine Assay were obtained, sandwich immunoassays were developed and their performance characterized and combined to develop multiplex assay panels as shown for the Canine Assay; [2] Method Development: Individual assays were then optimized to eliminate cross-reactivity, enhance sensitivity and dynamic range; and [3] Preliminary Validation: Multiplex assay panels were then validated with (a) normal serum and plasma from healthy dogs (b) culture supernatants [C/S] from dog PBMCs stimulated with lipopolysaccharides [LPS]. Canine Assay—[Panel; Cytokine/chemokine; Analyte: GMCSF, IL-2, IL-4, IL-6, IL-7, IL-8, IL-10, IL-18, IFNγ, IP-10, TNF-α, MCP-1, IL-15, KC. Panel; Endocrine; Analyte: GLP-1, Glucagon, Insulin, Leptin. Panel; Adipokine; Analyte: Adiponectin, Resistin.] The results are shown in Table 3.

Results & Discussion: [1] Reagent Procurement/Characterization: 20 canine molecules covering different key areas of nutritional & therapeutic interest were selected as targets. Several antibody pairs specific for these molecules were screened. These included canine-specific antibodies and where canine-specific antibodies were not available, antibodies raised against murine, rodent or human molecules were screened. Antibody pairs that yielded the best signal with canine proteins were selected to develop bead based sandwich immunoassays [Luminex xMAP platform]. [2] Method Development: The assays selected from step [1] were further optimized for cross-reactivity, sensitivity and dynamic range to obtain the final multiplexed panels. After optimization there was negligible cross reactivity within the multiplex panels. Various concentrations of analytes in a ‘serum matrix’ were used to construct “Standard Curves.” An analysis of the standard curves for IL-4 & IL-18 [canine cytokine panel] and Leptin & Insulin [endocrine hormone panel] indicate that they have slightly lower sensitivity as compared with the rest of the analytes. Antibodies for IL-8 seem to give a higher background signal as compared to other cytokines. However, these assay characteristics will not interfere in measurements of physiological levels since the normal levels of most of these analytes is in the pg/ml range. [3] Validation: A preliminary validation of these multiplexed panels was carried out using normal serum/plasma. Table 3A summarizes the data on cytokines & chemokines [39 healthy dogs, German Shepard, Labrador Retriever, Schnauzer, Siberian Huskie, ages 2 to 6 yrs.]. Table 3B summarizes data on canine endocrine hormones in two serum samples containing inhibitors to prevent GLP-1 degradation. Table 3C summarizes data obtained from the adipokine panel [serum samples from 17 Beagle and Mongrel].

TABLE 3A
Cytokines in Normal Canine Serum & Plasma (conc. in pg/ml)

| | Serum | | | | Plasma | | | |
|---|---|---|---|---|---|---|---|---|
| | | | | % | | | | % |
| Cytokine | Median | Min | Max | Detectable | Median | Min | Max | Detectable |
| TNFa | 2.2 | ND | 885.5 | 79.5 | 1.1 | ND | 221.4 | 56.4 |
| GMCSF | 289.0 | 61.0 | 40112.0 | 100.0 | 151.0 | ND | 31654.0 | 100.0 |
| IL-4 | 306.3 | ND | 5020.4 | 69.2 | 226.0 | ND | 1859.0 | 66.7 |
| MCP-1 | 349.0 | 144.2 | 24484.2 | 100.0 | 161.7 | 68.4 | 6422.0 | 100.0 |
| KC | 620.3 | 94.0 | 1201.5 | 100.0 | 32.3 | ND | 828.1 | 94.9 |
| IL-8 | 395.5 | 9.6 | 19832.9 | 100.0 | 556.8 | 9.1 | 2292.4 | 100.0 |
| IFNγ | ND | ND | 1980.0 | 28.2 | ND | ND | 1264.0 | 10.3 |
| Leptin | 2024.5 | ND | 57755.0 | 71.8 | 1764.0 | ND | 17905.0 | 76.9 |
| IL-10 | ND | ND | 8744.0 | 23.1 | ND | ND | 5204.9 | 12.8 |
| IP-10 | 35.0 | 7.0 | 1292.0 | 100.0 | 21.0 | ND | 541.0 | 84.6 |
| Glucagon | 91.4 | 5.3 | 1080.8 | 100.0 | 35.4 | ND | 1525.4 | 84.6 |
| IL-02 | 157.0 | ND | 17472.0 | 69.2 | ND | ND | 63072.0 | 48.7 |
| IL-6 | ND | ND | 18955.0 | 53.8 | ND | ND | 12768.0 | 38.5 |
| IL-7 | 270.0 | 46.0 | 26273.0 | 97.4 | 155.0 | ND | 65201.0 | 100.0 |
| IL-15 | 166.0 | 27.0 | 79521.0 | 97.4 | 116.0 | ND | 22939.0 | 87.2 |
| IL-18 | 3703.0 | 508.0 | 96292.0 | 100.0 | 3191.0 | 353.0 | 89725.0 | 100.0 |
| Insulin | 864.1 | 280.4 | 12469.0 | 100.0 | 591.4 | 49.0 | 5946.1 | 100.0 |

TABLE 3B
Hormones in Normal Canine Serum Samples

| | Leptin | Glucagon | | Insulin |
|---|---|---|---|---|
| Sample | (ng/mL) | (pg/mL) | GLP-1 (pM) | (ng/mL) |
| Morgan | 10.12 | 148.37 | 80.94 | 1.30 |
| Stoli | ND | 94.96 | ND | 1.50 |

TABLE 3C
Adiponectin (Acrp30) and Resistin in Normal Canine Serum Samples

| | cAcrp30 | cResistin |
|---|---|---|
| N = 17 | (ng/ml) | (ng/ml) |
| Min | 3.08 | 5.10 |
| Max | 32.37 | 253.05 |
| Median | 14.90 | 15.03 |

Experiments were also conducted to confirm that these panels detected cytokines secreted by PBMCs following stimulation with LPS. Representative data are shown in FIG. 2. A LPS-dose and time dependent secretion of IL-6, TNF-α, IL-8 & IL-18 can be clearly observed.

Conclusions: A multiplexed panel capable of measuring 20 different canine proteins in serum, plasma & tissue culture supernatants has been successfully developed. Panel includes assays for several proteins for which assays were previously not available. Preliminary validation of panel with normal serum/plasma samples demonstrates that panel is capable of measuring these molecules in serum/plasma. Assays carried out on culture supernatants from PBMCs treated LPS demonstrates that assays in panel is capable of detecting cytokines expected to be secreted in response to LPS stimulation, namely IL-6, TNF-α, IL-8 and IL-18 in a time as well as dose dependent manner. Further validation experiments are currently underway with appropriate nutrition & clinical studies. In conjunction with appropriate pattern recognition and pathway analysis techniques these panels will help predict/evaluate functional outcomes of physiological stressors and interventions.

It should be understood that various changes and modifications to the preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, is relevant prior art for the present invention and the right to challenge the accuracy and pertinence of such patents, patent applications, publications, and other references is specifically reserved.

What is claimed is:

1. A collection of detectable molecular probes for determining in a single sample, the activity, presence, or expression of each of a predetermined set of analytes, the set comprising at least one cytokine or gene therefor, one chemokine or gene therefor, one hormone or gene therefor, and one adipokine or gene therefor; wherein for each analyte in the set, the collection of molecular probes comprises at least one probe suitable for detecting the activity, presence, or expression of that analyte.

2. The collection of molecular probes of claim 1 wherein the set of analytes further comprises one or more neuronal growth factors or genes therefor, growth factors other than neuronal growth factors or genes therefor, soluble receptors or genes therefor, or combination thereof.

3. The collection of molecular probes of claim 2 comprising detectable probes for detecting an encoded gene product of each gene in the set of analytes thereby determining the expression of each gene in the set.

4. The collection of molecular probes of claim 3 wherein each probe is specific for detecting the one analyte in the set.

5. The collection of molecular probes of claim 3 wherein the detectable probes comprise antibodies, antibody fragments, ligands, receptors, binding proteins, or nucleic acids.

6. The collection of molecular probes of claim 1 wherein the set of analytes comprises one or more of the cytokines interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III, or a gene encoding any of the foregoing cytokines.

7. The collection of molecular probes of claim 6 wherein the set of analytes comprises one or more of the chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1, or a gene encoding any of the foregoing.

8. The collection of molecular probes of claim 7 wherein the set of analytes comprises one or of the hormones prolactin, insulin-like growth factor binding protein 2, leptin, insulin, resistin, adiponectin, glucagon, glucagon-related peptide 1, or PYY, or a gene encoding any of the foregoing.

9. The collection of molecular probes of claim 8 wherein the set of analytes comprises one or of the adipokines monocyte chemotactic protein 1, leptin, resistin, adiponectin, IL-6, TNF-alpha, or thrombin-activatable fibrinolysis inhibitor, or a gene encoding any of the foregoing.

10. The collection of molecular probes of claim 9 wherein the predetermined set of analytes further comprises one or more of the neuronal growth factors ciliary neurotrophic factor, glial cell line derived neurotrophic factor, brain-derived neurotrophic factor, neurotrophin 3, neurotrophin 4, or beta-nerve growth factor, or a gene encoding any of the foregoing.

11. The collection of molecular probes of claim 9 wherein the predetermined set of analytes further comprises one or more of the growth factors angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2, or a gene encoding any of the foregoing.

12. The collection of molecular probes of claim 9 wherein the predetermined set of analytes further comprises one or more of the soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cyotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4, or a gene encoding any of the foregoing.

13. The collection of molecular probes of claim 9 that comprises a specific probe for each of IL-2, IL-4, IL-6, IL-7, IL-8, IL-10, IL-18, IFN γ, IP-10, TNF-α, MCP-1, GLP-1, glucagon, insulin, adiponectin, and resistin.

14. The collection of molecular probes of claim 13 that are capable of detecting the activity, presence, or expression of each of a predetermined set of analytes from an animal that is human, murine, simian, canine, or feline.

15. The collection of molecular probes of claim 14 further comprising specific probes for detecting the presence or activity of one or more of IL-15, KC, and leptin, or a gene encoding any of the foregoing.

16. The collection of molecular probes of claim 13 wherein the analytes are from a canine and the probes are antibodies.

17. The collection of molecular probes of claim 16 which provide a quantitative determination of the activity, presence, or amount of expression of each analyte.

18. The collection of molecular probes of claim 17 wherein each probe is attached to a matrix, wherein each such attached probe remains capable of providing a quantitative determination of the activity, presence, or amount of expression of each analyte in a sample brought into contact with the matrix.

19. The collection of molecular probes of claim 18 wherein each probe is attached to a separate matrix.

20. The collection of molecular probes of claim 19 wherein each probe is independently detectable from each other probe in the collection.

21. A method of assessing the health status of an animal by determining the relative activity or expression of a set of genes, the method comprising the steps of
obtaining a biological sample from the animal; said sample putatively containing a predetermined set of analytes of interest or the expression products of those analytes, the set comprising at least of a cytokine, a chemokine, a hormone, and an adipokine, or a gene encoding each of the foregoing;
contacting the sample with a collection of molecular probes for determining the activity, presence, or expression of each of the predetermined set of analytes, wherein for each analyte in the set, the collection of molecular probes comprises at least one probe suitable for detecting the activity, presence, or expression of that analyte, each probe capable of producing an independently detectable signal when the analyte or expression product thereof corresponding to that probe is present in the sample;
detecting the independently detectable signals produced after the sample is contacted with the collection, correlating the detectable signals with the relative activity, presence, or expression of each of the predetermined set of analytes in the sample;
correlating the relative activity, presence, or expression of each of the predetermined set of analytes in the sample with known parameters of health status; and
making a determination of the health status of the animals in accordance therewith.

22. The method of claim 21 wherein the set of analytes further comprises one or more of a neuronal growth factor, a growth factor other than a neuronal growth factor, a soluble receptor, or a combination thereof, or a gene encoding any of the foregoing.

23. The method of claim 22 wherein the detectable probes are specific for detecting the presence, activity or expression of each analyte in the set of analytes.

24. The method of claim 23 wherein the detectable probes are antibodies, antibody fragments, ligands, receptors, or binding proteins.

25. The method of claim 21 wherein the set of analytes comprises one or more of the cytokines interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III, or a gene encoding any of the foregoing.

26. The method of claim 25 wherein the predetermined set of analytes includes one or more of the chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1, or a gene encoding any of the foregoing.

27. The method of claim 26 wherein the predetermined set of analytes includes one or more of the hormones prolactin, insulin-like growth factor binding protein 2, leptin, insulin, resistin, adiponectin, glucagon, glucagon-related peptide 1, or PYY, or a gene encoding any of the foregoing.

28. The method of claim 27 wherein the predetermined set of analytes includes one or more of the adipokines monocyte chemotactic protein 1, leptin, resistin, adiponectin, IL-6, TNF-alpha, or thrombin-activatable fibrinolysis inhibitor, or a gene encoding any of the foregoing.

29. The method of claim 28 wherein the predetermined set of analytes further comprises one or more of the neuronal growth factors ciliary neurotrophic factor, glial cell line derived neurotrophic factor, brain-derived neurotrophic factor, neurotrophin 3, neurotrophin 4, or beta-nerve growth factor, or a gene encoding any of the foregoing.

30. The method of claim 29 wherein the predetermined set of analytes further comprises one or more of the growth factors angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2, or a gene encoding any of the foregoing.

31. The method of claim 30 wherein the predetermined set of analytes further comprises one or more of the soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cyotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4, or a gene encoding any of the foregoing.

32. The method of claim 21 wherein the predetermined set of analytes comprises each of IL-2, IL-4, IL-6, IL-7, IL-8, IL-10, IL-18, IFN γ, IP-10, TNF-α, MCP-1, GLP-1, glucagon, insulin, adiponectin, and resistin, or at least one gene each encoding each of the foregoing.

33. The method of claim 32 wherein the animal is human, murine, simian, canine, or feline.

34. The method of claim 33 wherein the predetermined set of analytes further comprises one or more of IL-15, KC, or leptin, or a gene encoding any of the foregoing.

35. The method of claim 33 wherein the analytes are from a canine and the probes are antibodies.

36. The method of claim 35 wherein the collection of molecular probes allow for a quantitative determination of the activity, presence, or amount of expression of each analyte.

37. The method of claim 36 wherein each probe is attached to a matrix, wherein each such attached probe remains capable of providing a quantitative determination of the activity, presence, or amount of expression of an analyte corresponding to that probe, in a sample brought into contact with the matrix.

38. The method of claim 37 further comprising contacting the sample and the collection of molecular probes with a set of secondary antibodies comprising one or more antibodies to aid in the detection by increasing specificity or detection signal.

39. The method of claim 37 wherein each probe is attached to a separate matrix.

40. The method of claim 39 wherein the sample is serum or plasma.

41. A method of formulating a nutritional regimen for improving the health of an animal comprising
a) selecting a predetermined set of analytes of interest in an animal wherein the activity, presence, or expression of the analytes can be correlated with the health status of the animal, and with a nutritional regimen of the animal, the set comprising at least one each of a cytokine, a chemokine, a hormone, and an adipokine, or a gene encoding any of the foregoing;
b) obtaining a biological sample from the animal; said sample putatively containing the predetermined set of analytes, or the expression products thereof, said sample indicative of a current nutritional regimen of the animal;
c) determining a baseline measurement by contacting the sample with a collection of molecular probes for determining the activity, presence, or expression of each of the predetermined set analytes, wherein for each analyte in the set, the collection of molecular probes comprises at least one probe suitable for detecting the activity, presence, or expression of that analyte, each probe capable of producing an independently detectable signal when the analyte or expression product thereof corresponding to that probe is present in the sample;
d) detecting the independently detectable signals produced after the sample is contacted with the collection,
e) correlating the detectable signals with the relative activity, presence, or expression of each of the predetermined set of analytes in the sample;
f) correlating the relative activity, presence, or expression of each of the predetermined set of analytes in the sample with known parameters of health status;
g) making a determination of the health status of the animal on the current nutritional regimen in accordance therewith;
h) formulating one or more test nutritional regimens or supplements for testing in the animal by adjusting one or more of the macronutrient content or source, the micronutrient content or source, supplemental dietary components, or caloric content of the diet as compared to the current nutritional regimen of the animal;
i) providing one or more test nutritional regimens or supplements, or combination thereof, to the animal in an amount and for a time effective to change the activity, presence, or expression of one or more of the predetermined set of analytes;
j) for each test nutritional regimen or supplement, or combination thereof, repeating steps b) through
g) with a new sample from the animal to determine whether the test nutritional regimen or supplement, or combination thereof has improved the health status of the animal; and
k) selecting a formulated nutritional regimen or supplement, or combination thereof, which improves the heath status of the animal.

42. The method of claim 41 wherein the animal is obese, has diabetes, has symptoms of being predisposed to diabetes, has an undesirable level of inflammation, has an undesirable level of insulin resistance, has metabolic syndrome, premature atherosclerosis, abnormal glucose metabolism, or abnormal fat metabolism.

43. The method of claim 42 wherein the formulated nutritional regimen, supplement, or combination thereof reduces inflammation, reduces insulin resistance, or a combination thereof in the animal.

44. The method of claim 43 wherein the formulated nutritional regimen, supplement, or combination thereof reduces inflammation in the animal, and one or more of increases anti-inflammatory cytokines, reduces pro-inflammatory cytokines, or decreases cytokine mediators of inflammation.

45. The method of claim 44 wherein the pro-inflammatory cytokines include one or more of interferon alpha, interferon gamma, interleukin 12 p40, interleukin 18, interferon beta, interferon omega, lymphotoxin beta R, lymphotoxin, interleukin 6, interleukin 8, or tumor necrosis factor alpha; and wherein the anti-inflammatory cytokines include interleukin 4, interleukin 10, interleukin 13, transforming growth factor beta-1, or tumor necrosis factor beta

46. The method of claim 44 wherein the pro-inflammatory cytokines are IL-6 or IL-12, IFN, and the anti-inflammatory cytokines are IL-10 and IL-13.

47. The method of claim 42 wherein the formulated nutritional regimen, supplement, or combination thereof reduces insulin resistance in the animal and one or more of increases adiponectin, decreases resistin, or decreases leptin.

48. The method of claim 42 wherein the formulated nutritional regimen, supplement, or combination thereof reduces one or more of dyslipidemia, inflammation, hypertension, altered vascular reactivity, or visceral obesity, or improves fibrinolysis.

49. The method of claim 41 wherein the set of analytes further comprises one or more of a neuronal growth factor, a growth factor other than a neuronal growth factor, a soluble receptor, or combination thereof, or a gene encoding any of the foregoing.

50. The method of claim 41 wherein the detectable probes are specific for detecting each of the set of analytes.

51. The method of claim 41 wherein the detectable probes are antibodies, antibody fragments, ligands, receptors, or binding proteins.

52. The method of claim 41 wherein the set of analytes comprises one or more of the cytokines interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III, or a gene encoding any of the foregoing.

53. The method of claim 52 wherein the predetermined set of analytes includes one or more of the chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein 1 delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1, or a gene encoding any of the foregoing.

54. The method of claim 53 wherein the predetermined set of analytes includes one or more of the hormones prolactin, insulin-like growth factor binding protein 2, leptin, insulin, resistin, adiponectin, glucagon, glucagon-related peptide 1, or PYY, or a gene encoding any of the foregoing.

55. The method of claim 54 wherein the predetermined set of analytes includes one or more of the adipokines monocyte chemotactic protein 1, leptin, resistin, adiponectin, IL-6, TNF-alpha, or thrombin-activatable fibrinolysis inhibitor, or a gene encoding any of the foregoing.

56. The method of claim 55 wherein the predetermined set of analytes further comprises one or more of the neuronal growth factors ciliary neurotrophic factor, glial cell line derived neurotrophic factor, brain-derived neurotrophic factor, neurotrophin 3, neurotrophin 4, or beta-nerve growth factor, or a gene encoding any of the foregoing.

57. The method of claim 56 wherein the predetermined set of analytes further comprises one or more of the growth factors angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2, or a gene encoding any of the foregoing.

58. The method of claim 57 wherein the predetermined set of analytes further comprises one or more of the soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cyotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4, or a gene encoding any of the foregoing.

59. The method of claim 41 wherein the predetermined set of analytes comprises each of IL-2, IL-4, IL-6, IL-7, IL-8, IL-10, IL-18, IFN γ, IP-10, TNF-α, MCP-1, GLP-1, glucagon, insulin, adiponectin, and resistin, or at least one gene each encoding each of the foregoing.

60. The method of claim 59 wherein the animal is human, murine, simian, canine, or feline.

61. The method of claim 60 wherein the predetermined set of analytes further comprises one or more of IL-15, KC, or leptin, or a gene encoding any of the foregoing.

62. The method of claim 60 wherein the analytes are from a canine and the probes are antibodies.

63. The method of claim 62 wherein the collection of molecular probes allow for a quantitative determination of the activity, presence, or expression of each analyte.

64. The method of claim 63 wherein each probe is attached to a matrix, wherein each such attached probe remains capable of providing a quantitative determination of the activity, presence, or amount of expression of an analyte corresponding to that probe, in a sample brought into contact with the matrix.

65. The method of claim 64 comprising the further step of contacting the sample and the collection of molecular probes with a set of secondary antibodies comprising one or more antibodies to aid in the detection by increasing specificity or detection signal.

66. The method of claim 65 wherein each probe is attached to a separate matrix.

67. The method of claim 66 wherein the sample is tissue or bodily fluid.

68. The method of claim 67 wherein the fluid is serum or plasma.

69. A collection of detectable molecular probes for determining in a single sample, the presence of a predetermined set of protein analytes, the set comprising a specific probe for each of IL-2, IL-4, IL-6, IL-7, IL-8, IL-10, IL-18, IFN γ, IP-10, TNF-α, MCP-1, GLP-1, glucagon, insulin, adiponectin, and resistin; wherein each probe is an antibody or antibody fragment specific for detecting one protein in the set, each probe being independently detectable from each other probe in the collection, each such probe attached to a matrix, wherein each attached probe is capable of providing a quantitative determination of the corresponding protein in the set, in a sample brought into contact with the matrix; wherein the proteins are encoded by genes from an animal that is human, murine, simian, canine, or feline.

70. The collection of molecular probes of claim 68 further comprising specific probes for one or more of IL-15, KC, and leptin.

71. The collection of molecular probes of claim 69 wherein the set of protein analytes further comprises:
one or more of the cytokines interferon alpha, interferon gamma, interleukin 12 p40, interleukin 18, interferon beta, interferon omega, lymphotoxin beta R, lymphotoxin, interleukin 6, interleukin 8, tumor necrosis factor alpha, interleukin 4, interleukin 10, transforming growth factor beta-1, tumor necrosis factor beta, interleukin 3, interleukin 5, interleukin 7, interleukin 13, interleukin 15, interleukin 1 alpha, interleukin 1 beta, interleukin 2, interleukin 11, interleukin 12 p70, interleukin 16, interleukin 17, Regulated upon Activation, Normal T Expressed and presumably Secreted (RANTES), interleukin 21, interleukin 9, or transforming growth factor beta receptor III; and
one or more of the chemokines B-lymphocyte chemoattractant, epithelial cell-derived neutrophil-activating peptide, eotaxin, eotaxin-2, monocyte chemotactic protein 2, monocyte chemotactic protein 3, macrophage migration inhibitory factor, macrophage inflammatory protein 1 alpha, myeloid progenitor inhibitory factor 1, macrophage stimulating protein, granulocyte chemotactic protein 2, interferon gamma inducible protein 10, leukemia inhibitory factor, macrophage colony stimulating factor, monocyte chemotactic protein 1, macrophage-derived chemokine, macrophage inflammatory protein 1 beta, macrophage inflammatory protein I delta, neutrophil activating peptide 2, pulmonary- and activation-regulated chemokine, stromal cell-derived factor alpha, thymus- and activation-regulated chemokine, betacellulin, 6 Ckine, fibroblast growth factor acidic, fractalkine, hemofiltrate CC chemokine 1, monocyte chemotactic protein 4, macrophage inflammatory protein 3 beta, platelet factor 4, receptor Activator of NF-kappa-B, cutaneous T-cell attracting chemokine, eotaxin-3, fibroblast growth factor-4, follistatin, growth-related oncogene gamma, interferon gamma-inducible T cell alpha chemoattractant, leukemia inhibitory factor receptor alpha, midkine, macrophage inflammatory protein 3 alpha, pleiptrophin, stromal cell-derived factor beta, thymus-expressed chemokine, transforming growth factor alpha, TNF-related activin-induced cytokine, vascular adhesion protein-1, CXCL9, or CCL1; and
one or more of the hormones prolactin, insulin-like growth factor binding protein 2, leptin, insulin, resistin, adiponectin, glucagon, glucagon-related peptide 1, or PYY; and
one or more of the adipokines monocyte chemotactic protein 1, leptin, resistin, adiponectin, IL-6, TNF-alpha, or thrombin-activatable fibrinolysis inhibitor; and
one or more of:
the neuronal growth factors ciliary neurotrophic factor, glial cell line derived neurotrophic factor, brain-derived neurotrophic factor, neurotrophin 3, neurotrophin 4, or beta-nerve growth factor; or
the growth factors angiogenin, epidermal growth factor, fibroblast growth factor-7, fibroblast growth factor-9, granulocyte macrophage colony stimulating factor, melanoma growth-stimulating activity, oncostatin M, placenta growth factor, transforming growth factor beta-3, amphiregulin fibroblast growth factor-6, granulocyte colony stimulating factor, stem cell factor, vascular endothelial growth factor, cardiotrophin-1, growth-related oncogene beta, heparin-binding EGF-like growth factor, hepatocyte growth factor, herpesvirus entry mediator, matrix metalloproteinase 10, matrix metalloproteinase 7, matrix metalloproteinase 9, tissue inhibitors of metalloproteinases 1, vascular endothelial growth factor D, vascular endothelial growth factor receptor 2, fibroblast growth factor basic, insulin-like growth factor I, insulin-like growth factor II, insulin-like growth factor binding protein 1, Insulin-like Growth Factor Binding Protein 3, Insulin-like Growth Factor Binding Protein 4, Insulin-like Growth Factor Binding Protein 6, Matrix Metalloproteinase 1, Matrix Metalloproteinase 2, or Tissue Inhibitor of Metalloproteinases 2; or
the soluble receptors sCD23, Fas (CD95), interleukin 1 receptor antagonist, interleukin 2 soluble receptor alpha, TNF-related apoptosis inducing ligand, urokinase-type plasminogen activator receptor, fms-like tyrosine kinase-3 ligand, soluble glycoprotein 130, interleukin 1 soluble receptor I, interleukin 6 soluble receptor, tumor necrosis factor receptor I, tumor necrosis factor receptor II, vascular epithelium cadherin, CCL28, cyotoxic T-lymphocyte-associated molecule 4, death receptor 6, Fas ligand, intercellular adhesion molecule 3, interleukin 2 receptor gamma, interleukin 5 receptor alpha, L-selectin, platelet endothelial cell adhesion molecule-1, Stem Cell Factor Receptor, TNF-related Apoptosis-inducing Ligand Receptor 4, Activated Leukocyte Cell Adhesion, CD27, CD30, CD40, ciliary neurotrophic factor receptor, Intercellular Adhesion Molecule 1, Insulin-like Growth Factor I Receptor, Interleukin 1 soluble receptor II, interleukin 2 receptor beta, Interleukin 10 receptor beta, Macrophage colony stimulating factor receptor, Platelet-derived Growth factor Receptor alpha, or TNF-related Apoptosis-inducing Ligand Receptor 4.

72. A kit suitable for determining in a single sample, the activity, presence, or expression of each of a predetermined set of analytes comprising in separate containers in a single package or in separate containers in a virtual package, as appropriate for the kit component, a multiplex assay comprising a collection of detectable molecular probes and one or more of (1) instructions for how to use the multiplex assay to determine the activity, presence, or expression of each of a predetermined set of analytes, (2) instruction for how to assess the health status of an animal using the multiplex assay, (3) instructions for formulating a nutritional regimen for improving the health of an animal using the multiplex assay, and (4) one or more ingredients suitable for consumption by an animal.

73. A means for communicating information about or instructions for using the multiplex assay for one or more of (1) determining in a single sample, the activity, presence, or expression of each of a predetermined set of analytes, (2) assessing the health status of an animal, or (3) formulating a nutritional regimen for improving the health of an animal comprising a document, digital storage media, optical storage media, audio presentation, or visual display containing the information or instructions.

74. The means of claim 73 selected from the group consisting of a displayed web site, visual display kiosk, brochure, product label, package insert, advertisement, handout, public announcement, audiotape, videotape, DVD, CD-ROM, computer readable chip, computer readable card, computer readable disk, computer memory, or combination thereof.
